(19) United States
(12) Patent Application Publication
(10) Pub. No.: US 2002/0173479 A1
(43) Pub. Date: Nov. 21, 2002

(54) Methods for the treatment and diagnosis of prostate cancer based on p75NTR tumor supression

(76) Inventor: Daniel Djakiew, McLean, VA (US)

Correspondence Address: PILLSBURY WINTHROP, LLP, P.O. BOX 10500, MCLEAN, VA, 22102, US

(21) Appl. No.: 10/071,648

(22) Filed: Feb. 11, 2002

Related U.S. Application Data
(60) Provisional application No. 60/268,940, filed on Feb. 16, 2001.

(51) Int. Cl. A61K 48/00; A61K 9/127

(52) U.S. Cl. 514/44; 424/450; 435/320.1

(57) ABSTRACT

The present invention provides a method of treatment or prophylaxis of cancer in a subject in need thereof including administering to the subject p75^{NTR} gene or a fragment thereof in an amount effective to increase tumor suppression and/or tumor apoptosis. The p75^{NTR} gene or fragment thereof is administered in an amount sufficient to maintain a level of p75^{NTR} mRNA which at least partially compensates for the loss of p75^{NTR} mRNA associated with p75^{NTR} mRNA degradation in cancerous or precancerous cells. The invention also includes administering to the subject a p75^{NTR} mRNA stabilizing agent (e.g., one or more RNA-binding proteins).

### CROSS-REFERENCE TO RELATED APPLICATIONS

The present application claims priority to U.S. Provisional Application Serial No. 60/268,940 filed Feb. 16, 2001, the contents of which are hereby incorporated by reference in their entirety.

### BACKGROUND OF THE INVENTION

1. Field of the Invention

The present invention relates to the diagnosis and treatment of prostate cancer. More particularly, the invention relates to the treatment of cancer by promotion of the expression of the p75^{NTR} gene.

1. Summary of the Related Art

The prostate is the most frequent site of cancer diagnosis and second leading site of cancer mortality in men of the combined countries of Western origin (Landis, 1998). Prostate cancer is also the most common malignancy after ovarian and breast cancer kindred's segregated by chromosome 17q21 (2, 3). This suggests that gene(s) in the immediate vicinity of 17q21 are important in the development of prostate cancer (4). Direct experimental studies using microcell mediated chromosomal transfer has identified a tumor suppressor gene associated with prostate cancer in the region 17q12-q22 (5). Moreover, a high frequency loss of heterozygosity in prostate cancer has been detected in the vicinity of 17q21 (4, 6). Although the BRCA1 tumor suppressor gene has been localized to this region, not all of the prostate tumor suppressor activity in the region of 17q21 can be fully accounted for by the BRCA1 gene (6). Hence, it has been suggested that another unidentified tumor suppressor gene in this region may be important in the development of prostate cancer (6), and that BRCA1 itself plays only a minor role in prostate cancer development (7).

Interestingly, the human p75^{NTR} gene locus has been mapped closely distal to 17q21 (8). p75^{NTR} is a 75 kDa glycoprotein receptor that binds the neurotrophin family of growth factors, including nerve growth factor, brain-derived neurotrophic factor, neurotrophin-3 and neurotrophin-4/5. Expression of the p75^{NTR} protein as studied by immunoblot techniques (9), immunofluorescence (10), immunohistochemistry (12) and Scatchard plot analysis (12) have all shown a decline of this receptor with progression of the prostate to cancer. Loss of expression of p75^{NTR} protein is correlated with increased Gleason's score of organ confined pathological prostate tissues (13), and is completely absent from four prostate epithelial tumor cell lines derived from metastases (9), indicating an inverse association of p75^{NTR} expression with the malignant progression of the prostate. The significance of a loss of expression of p75^{NTR} protein during malignant transformation of prostate epithelial cells may be related to observations that this receptor appears to function in the induction of apoptosis (14, 15). Re-expression of p75^{NTR} by stable and transient transfection showed that the p75^{NTR} inhibits growth of prostate tumor cells in vitro, at least in part, by induction of apoptosis [16]. Hence, loss of p75^{NTR} expression appears to eliminate a potential apoptotic pathway in prostate cancer cells, thereby facilitating the immortalization of these epithelia during malignant transformation [13]. Considering the characterization of a prostate tumor suppressor gene locus in the vicinity of the p75^{NTR} gene, the inverse association of p75^{NTR} expression with the malignant progression of the pathologic prostate, and transfection studies showing that p75^{NTR} can induce apoptosis in vitro, we formally investigated whether the p75^{NTR} is a new tumor suppressor in the human prostate [13].

The low affinity nerve growth factor receptor p75^{NTR} belongs to the tumor necrosis factor receptor super-family and has been implicated in induction of apoptosis in various tissues and cell lines. p75^{NTR} is a 75-kDa glycoprotein that binds nerve growth factor and has structural and sequential similarity to the tumor necrosis factor receptor (Chao et al., 1986, Radeke et al., 1987). This similarity suggests a role in apoptosis which was demonstrated in neuronal cells (Lee et al., 1994, Frade et al., 1996). Normal prostate and prostatic intraepithelial neoplastic tissue exhibit staining of p75^{NTR} in all epithelial cells, while in neoplastic prostate the epithelial cells exhibit a partial loss of p75^{NTR} expression (Perez et al., 1997). Western blot analysis of the four naturally occurring human prostate tumor cell lines TSU-pr1, DU-145, PC-3, and LNCaP, derived from metastases, shows that there is a complete loss of p75^{NTR} protein expression (Pflug et al., 1992), as later confirmed by Scatchard plot analysis (Pflug et aL., 1995). Subsequent transfection and re-expression of the p75^{NTR} protein in prostate tumor cells showed a role in the induction of apoptosis (Pflug and Djakiew, 1998). Hence, loss of p75^{NTR} expression in prostate tumor cells was suggested as a mechanism by which tumor cells circumvented apoptotic inhibition of tumor cell growth (Pflug and Djakiew, 1998). However, it is not known whether this loss of expression is due to deletion of part or the entire p75^{NTR} gene, or to other factor(s).

Thus, there remains a need for the elucidation of the mechanisms through which the p75^{NTR} gene plays a role in prostate cancer and the design of therapeutic and diagnosis protocols based on the elucidation of those mechanisms.

### SUMMARY OF THE INVENTION

The present invention provides a method of treatment or prophylaxis of cancer in a subject in need thereof comprising administering to the subject p75^{NTR} gene or a fragment thereof in an amount effective to increase tumor suppression and/or tumor apoptosis. Preferably, the p75^{NTR} gene or fragment thereof is administered in an amount sufficient to maintain a level of p75^{NTR} mRNA which at least partially compensates for the loss of p75^{NTR} mRNA associated with p75^{NTR} mRNA degradation in cancerous or precancerous cells. The method of the invention is particularly effective in the treatment of prostate cancer.

The invention also provides a method of treatment or prophylaxis of cancer in a subject in need thereof comprising administering to the subject a p75^{NTR} mRNA stabilizing agent such as one ore more RNA-binding protein.

Also provided is a method for early diagnosis of prostate cancer comprising determining p75^{NTR} mRNA levels in prostate tissue of a subject. In one embodiment of the invention, determining p75^{NTR} mRNA levels in prostate tissue comprises isolating the RNA from the tissue; subjecting the RNA to reverse transcription and then to PCR amplification with a suitable primer; precipitating the product of the amplification reaction; and subjecting the precipitate to electrophoresis analysis to determine the level of RNA in the prostate tissue.

### BRIEF DESCRIPTION OF THE DRAWINGS

FIG. 1. Western blot of p75^{NTR} protein in Neo, Low (Low), Intermediate (Int), and High (High) expression clones of TSU-pr1 cells with A875 cells as a positive control. Detection of the p75^{NTR} protein was carried out through the use of antibody MAB5264 as described in Material and Methods. The location of the molecular weight markers is indicated to the left.

FIG. 2. Graph of the effect of p75^{NTR} protein expression (neo, low, intermediate, high) on the phases of the cell cycle of the TSU-pr1 clones. The cells were washed in serum-free DMEM, and incubated for 24 hours in serum-free DMEM at 37° C., stained with propidium iodide, and subjected to fluorescence-activated cell sorter (FACS) cell cycle analysis as described in Material and Methods. Bars represent the mean of six independent experiments ± standard error. *p<0.000001.

FIG. 3. Graph of the effect of p75^{NTR} protein expression (neo, low, intermediate, high) on tumor growth of the TSU-pr1 clones. Cells (1×10⁶) were injected subcutaneously per site, with 20 sites per group. The tumors were measured twice a week and the volume was calculated by the formula ┌┐/6xLxWxH. Points on the graph represent the mean of the tumor volume for each group at the specified day. The graph is representative of four independent experiments. *p<0.05, **p<0.0005, ***p<0.00005.

FIG. 4. Representative tumors formed from TSU-pr1 clones of neo (A), low (B), intermediate (C) and high (D) p75^{NTR} expression cells in both flanks of SCID mice. Cells (1×10⁶) were injected subcutaneously into the flanks of SCID mice and allowed to grow for 24 days.

FIG. 5. Graph of the effect of p75^{NTR} protein expression on the percentage of cells undergoing programmed cell death within the SCID mice tumors. The tumors were sectioned, de-paraffinized and stained by the TUNEL technique as described in Material and Methods. The percentage of cells undergoing apoptosis was calculated by dividing the number of TUNEL positive cells by the total number of cells. A total of 1600-1800 cells were counted per group and each group was counted three times to obtain a mean percentage of cells that stain positive for TUNEL. Bars represent the mean of three cell counts ± standard error. *p<0.05, **p<0.005, ***p<0.0005.

FIG. 6. Graph of the effect of p75^{NTR} protein expression on PCNA staining within the SCID mice tumors. The tumors were sectioned, de-paraffinized and stained for PCNA expression as described in Material and Methods. The percentage of proliferating cells was calculated by dividing the number of PCNA positive cells by the total number of cells. A total of 3000-3300 cells were counted per group and each group was counted three times to obtain a mean percentage of cells that stain positive for PCNA. Bars represent the mean of three cell counts ± standard error. *p<0.005,** p<0.000005.

FIG. 7. Southern blot analysis (A and B) of genomic DNA from A875 (A), LNCaP (L), TSU-pr1 (T), DU-145 (D), and PC-3 (P) cell lines were digested with either EcoRI (denoted by subscript E) or BamHI (denoted by subscript B).

FIG. 8. PCR of p75^{NTR} exons 1 (A), 4 (B), and 6 (C) of genomic DNA from A875 (A), DU-145 (D), PC-3 (P), LNCaP (L), and TSU-pr1 (T) cell lines, and the marker is denoted by M. The left panels are ethidium bromide stained gels, and the right panels are the same gels subjected to Southern blot analysis.

FIG. 9. RT-PCR analysis of mRNA extracted from A875 (A), DU-145 (D), PC-3 (P), LNCaP (L), and TSU-pr1 (T) cell lines.

FIG. 10. RNase protection of mRNA from A875 (A), DU-145 (D), PC-3 (P), LNCaP (L), and TSU-pr1 (T) cell lines using a p75^{NTR} and a GAPDH probe.

FIG. 11. Western blot of transiently transfected DU-145 (D), TSU-pr1 (T), and PC-3 (P) cell lines using either pMVE1 plasmid (denoted by subscript F) or pCMV5A (denoted by subscript T).

FIG. 12. PCR of genomic DNA from transiently transfected DU-145 (D), TSU-pr1 (T), and PC-3 (P) cell lines using either pMVE1 plasmid (denoted by subscript F) or pCMV5A (denoted by subscript T) run on an ethidium stained gel.

FIG. 13. Photographs of TSU-pr1 tumors grown subcutaneously in SCID mice treated with 100 ng/ml NGF. NGF stimulated the formation of small tumors contiguous (arrows) with the main tumor mass (a & b) and small non-contiguous tumors that occurred at some distance (arrow heads) from the main tumor mass (b).

FIG. 14. Diagram of the death receptor signal transduction cascade. A cytoplasmic death receptor domain can initiate signaling via NFκB and/or JNK.

FIG. 15. Western blot of death receptor signaling proteins in PC-3 cancer cells, categorized in rank-order as neo control (N), low (L) and high (H) expressors of the p75^{NTR} protein, and TSU-pr1 cancer cells, categorized in rank-order as neo control (N), low (L), intermediate (I) and high (H) expressors of the p75^{NTR} protein, in vitro.

FIG. 16. Western blots of transfected tumors cells, categorized in rank-order as neo control, low, intermediate (int.) and high expressors of the p75^{NTR} protein, and the corresponding levels of components of the cyclin/cdk complexes in these clones.

FIG. 17. Activity of CDK2 in tumor cells, categorized in rank-order as neo control, low, intermediate (int.) and high expressors of the p75^{NTR} protein.

FIG. 18. Western blots of transfected tumors cells, categorized in rank-order as neo control, low, intermediate (int.) and high expressors of the p75^{NTR} protein, and the corresponding levels of pRb, phosphorylated Rb (pRb-P), E2F and PCNA in these clones.

FIG. 19. Western blots of transfected tsu-pr1 prostate tumors cells, categorized in rank-order as neo control, low, intermediate (int.) and high expressors of the p75^{NTR} protein, and the corresponding levels of pro-apoptotic proteins, bad, bax, bid and bak, and the anti-apoptotic proteins, bcl-2, bcl-xl and phosphorylated bad (bad-p) in the same clones. it is clear that increasing p75^{NTR} protein expression was associated with increased pro-apoptotic effectors, and a reduction in pro-survival (anti-apoptotic) effectors.

FIG. 20. Time course (0-6 hrs) of cytochrome c release from mitochondria into the cytosol of tumor cells that do not express p75^{NTR} (neo) or have high expression of p75^{NTR} in the precense of cyclohexamide (CHX).

FIG. 21. Western blots of transfected tumors cells, categorized in rank-order as neo control, low, intermediate (int.) and high expressors of the p75^{NTR} protein, showing the presence of apaf-1, the reduced expression of IAP1, the 35 kDa form of procaspase-9 and its 10 kDa cleavage product, and the 35 kDa form of procaspase-7 and its 20 kDa cleavage product following activation in the absence (control) or presence of cyclohexamide (+CHX).

FIG. 22. Western blots of transfected tumors cells, categorized in rank-order as neo control, low, intermediate (int.) and high expressors of the p75^{NTR} protein, showing expression of procaspases-2,-3,-6,-8,-10 which were not activated in the presence of cyclohexamide.

FIG. 23. Hoechst staining of tumor cells that do not express p75^{NTR} (A, B), or express high levels of p75^{NTR} (C, D) in the absence (A, C) or presence of cyclohexamide (B, D).

FIG. 24. Gene therapy with the p75^{NTR} expression vector compared with liposome delivery vehicle alone (control) by intra-tumoral injection into PC-3 human prostate tumors grown in the flanks on SCID mice. *p<0.01

### DETAILED DESCRIPTION OF THE PREFERRED EMBODIMENTS

### I. MATERIAL AND METHODS

### Tissue Culture and Cell Lines

The prostate epithelial cell line TSU-pr1 was provided by Dr. John Issacs (Johns Hopkins University, Baltimore, Md.). The prostate epithelial cell lines PC-3, DU-145 and LNCaP were obtained from American Type Culture Collection (ATCC; Rockville, Md.). The A875 human melanoma cell line was provided by the laboratory of Dr. Moses Chao (Cornell University, New York, N.Y.). The cells were maintained in DMEM (Delbucco's Modified Eagles Medium; Mediatech Inc., Herndon, Va.) containing 4.5 g/L glucose and L-glutamine supplemented with antibiotic/antimycotic (100 units/ml penicillin G, 100 μg/mi streptomycin, 0.25 μg/ml amphotercin B; Mediatech Inc., Herndon, Va.) and 5% FBS (Sigma Chemical Co., St. Louis, Mo.). Media for the LNCaP cell line also contained 10⁻⁷ DHT. Media for the A875 cell line contained 10% FBS. All cell cultures were incubated at 37° C. in 10% CO₂/90% air. The p75^{NTR} transfected TSU-pr1 clones were previously described (16). The cells were maintained in DMEM (Dulbecco's Modified Eagles Medium; Mediatech Inc., Herndon, Va.) containing 4.5 g/L glucose and L-glutamine supplemented with antibiotic/antimycotic (100 units/ml penicillin G, 100 μg/ml streptomycin, 0.25 μg/ml amphotericin B; Mediatech Inc., Herndon, Va.) and 5% FBS (Sigma Chemical Co., St. Louis, Mo.) and 200 μg/ml G418 (Mediatech Inc., Herndon, Va.). All cell cultures were incubated at 37° C. in 10% CO₂/90% air.

### Southern Blot Analysis

Genomic DNA was isolated from various cell lines by treatment with trypsin-EDTA (Mediatech Inc., Herndon, Va.), followed by centrifugation at 500×g, rinsing with ice cold PBS (137 mM NaCl, 2.7 mM KCl, 4.3 mM Na₂HPO₄-7H₂O, 1.4 mM KH₂PO₄), centrifugation at 500×g, and then incubating the cells at 50° C. for 12 hours in digestion buffer (100 mM NaCl, 10 mM Tris-Cl (pH 8.0), 25 mM EDTA (pH 8.0), 0.5% sodium dodecyl sulfate, 0.1 mg/ml proteinase K (Sigma Chemical Co., St. Louis, Mo.)). The samples were extracted with equal volumes of phenol/chloroform/isoamyl alcohol, and centrifuged at 1700×g. The DNA was precipitated from the aqueous phase by adding half the volume of 7.5 M ammonium acetate and two volumes of 100% ethanol. The DNA was collected by centrifugation at 1700×g, washed with 70% ethanol, and resuspended in TE (10 mM Tris-HCl pH 8.0, 1 mM EDTA pH 8.0) buffer.

Genomic DNA (10-15 μg) were digested with 20 units per digestion of EcoRI or BamHI (New England Biolabs, Beverly, Mass.) at 37° C. for 4 hours. The digested DNA was run on a 0.8% agarose (Sigma Chemical Co., St. Louis, Mo.) gel in TAE (40 mM Tris-acetate, 2mM Na₂EDTA-2H₂O) buffer. The DNA in the gel was depurinated for 10 minutes in 0.2 N HCl solution followed by denaturation for 45 minutes in 1.5 M NaCl, 0.5 N NaOH, and neutralized for 30 minutes in 1 M Tris (pH 7.4), 1.5 M NaCl. The DNA was then transferred to Hybond N+(Amersham, Arlington Heights, Ill.) nylon membrane through capillary transfer in 10× SSC (3 M NaCl, 300 mM sodium citrate-2H₂O, pH 7.0). The DNA was crosslinked in a GS Gene Linker (Bio-Rad Laboratories, Hercules, Calif.) UV chamber.

The membrane was prehybridized for 2 hours at 65° C. in 5× Denhardt's (1 g Ficoll (Type 400), 1 g polyvinylpyrrolidone, 1 g bovine serum albumin), 6× SSC, 0.5% SDS and 100 μg/ml denatured, fragmented salmon sperm DNA. The p75^{NTR} radiolabeled probe was created using the High Prime DNA Labeling Kit (Roche Molecular Biochemicals, Indianapolis, Ind.) according to the manufacturer's instructions. Briefly, denatured DNA was added to High Prime reaction mixture along with dATP, dGTP, dTTP and [α³²P]dCTP (6000 Ci/mmol; Amersham Life Sciences, Inc., Arlington Heights, Ill.). The radiolabeled probe was then denatured and added to the prehybridization buffer and hybridization was undertaken for 16 hours at 65° C. After hybridization the membrane was washed in 2× SSC, 0.5% SDS for 15 minutes at room temperature, followed by 2-3 washes in 0.1× SSC, 0.5% SDS for 1 hour each at 68° C. The blot was exposed to Hyperfilm MP (Amersham Life Sciences, Inc., Arlington Heights, Ill.) autoradiography film and developed in a 100Plus Automatic X-ray Film Processor (All-Pro Imaging Corp., Hicksville, N.Y.).

### Western Blot

Protein was obtained from clones of the neo, low p75^{NTR} expression, intermediate p75^{NTR} expression and high p75^{NTR} expression TSU-pr1 cell lines after treating the cells in lysis buffer (10 mM Tris-HCl pH 7.4, 10 mM NaCl, 3 mM MgCl₂, 0.5% Igepal CA-630 (Sigma Chemical Co., St. Louis, Mo.), 2 μg/ml aprotinin (Sigma Chemical Co., St. Louis, Mo.) and 2 μg/ml leupeptin (Sigma Chemical Co., St. Louis, Mo.). Each protein sample (50 μg) was separated on a 10% sodium dodecyl sulfate-polyacrylamide gel as previously described (9) and transferred to nitrocellulose (Amersham Life Sciences, Inc., Arlington Heights, Ill.). The nitrocellulose was blocked in 5% non-fat milk in PBS for 1 hour, rinsed twice with TTBS (20 mM Tris-HCl, 500 mM NaCl, pH 7.5, 0.1% SDS), incubated overnight at room temperature with the murine monoclonal anti-human p75^{NTR} antibody MAB5264 (1:1000 dilution; Chemicon International, Inc., Temecula, Calif.) in TTBS. The blots were washed twice for 5 minutes each in TTBS and incubated with a horseradish peroxidase conjugated goat anti-mouse IgG (1:5000 dilution; Santa Cruz Biotechnology, Santa Cruz, Calif.) in TTBS for 1 hour at room temperature, rinsed twice in TTBS for 5 minutes each and finally for 5 minutes in TBS. Immunoreactivity was visualized with Opti-4CN (Bio-Rad, Richmond, Calif.).

### Cell Cycle Analysis

TSU-pr1 neo, low p75^{NTR} expression, intermediate p75^{NTR} expression and high p75^{NTR} expression cells were plated in growth medium in 10-cm culture plates and incubated at 37° C. in 10% CO₂/90% air until 30-40% confluent. The cells were then rinsed in serum-free DMEM, and synchronized by incubation in serum-free DMEM at 37° C. in 10% CO₂/90% air for 24 hours. After washing in PBS (×2), the cells were trypsinized, resuspended in growth medium, and counted. Two million cells per plate, with six plates per clone, were pelleted by centrifugation and resuspended in 100 μl citrate buffer (40 mM trisodium citrate-2H₂O, 250 mM sucrose, and 5% DMSO, pH 7.6). Nuclei were prepared for flow cytometric cell cycle analysis by Dr. Owen Blair and members of the Vincent T. Lombardi Cancer Research Center Flow Cytometry Core Facility (Georgetown University Medical Center, Washington, D.C.) using the method of Vindelov et al (17), with propidium iodide as the stain for nucleic acid. Cell cycle analysis was performed using the FACStar Plus fluorescence-activated cell sorter (Becton Dickinson Immunocytochemistry Systems, Mountainview, Calif.) equipped with the ModFit cell cycle analysis program (Verity Software House, Topsham, Me.).

### Tumor Growth.

TSU-pr1 neo, low p75^{NTR} expression, intermediate p75^{NTR} expression and high p75^{NTR} expression clones (1×10⁶ cells) were respectively injected subcutaneously into both flanks of 7 week old male ICR severe combined immunodeficient (SCID) mice (Taconic, Germantown, N.Y.) in combination with 10 μg/ml Matrigel (Becton Dickinson, Franklin Lakes, N.J.) to a total volume of 100 μl per injection site with twenty sites per group. Tumor lengths, widths, and heights were measured twice a week. Tumor volumes were calculated with the formula ┌┐6xLxWxH (18). Statistical differences between groups were determined by analysis of variance using GraphPad Prism 3.0 software (GraphPad Software, San Diego, Calif.).

### Immunohistochemistry

Tumors from neo, low p75^{NTR}, intermediate p75^{NTR} and high p75^{NTR} expression groups were collected upon sacrificing the mice and fixed in a 10% buffered formalin solution followed by embedding in paraffin wax. Tissue sections of 5 μm were de-paraffinized in three xylene washes of five minutes each, followed by immersion in a graded series of ethanol solutions (100%, 90%, 70%) for five minutes each and a final immersion in PBS for five minutes prior to any staining of the tissue sections. TUNEL staining was carried out using Apoptag (Intergen Company, Purchase, N.Y.) according to manufacturer's protocol, and proliferating cell nuclear antigen (PCNA) staining was carried out using the Zymed PCNA Staining Kit (Zymed Laboratories Inc., San Francisco, Calif.) according to the manufacturer's protocol. Random areas on the slides were counted for total number of cells, and cells that positively stained for either TUNEL or PCNA expression. For the TUNEL stained sections, a total of 1600-1800 cells per group were counted with each group counted three times independently by two investigators. For the PCNA stained sections, a total of 3000-3300 cells per group were counted with each group counted three times independently by two investigators. The percentage of cell nuclei that stained for either apoptosis (TUNEL) or proliferation (PCNA) was calculated by dividing the number of positive cell nuclei by the total number of cell nuclei.

### PCR of p75^{NTR} Exons 1,4, 6

For genomic investigations, the following primers were created: for exon 1 forward primer 5′- AAAGCTTACCGAGCTGGMG-3′ reverse primer 5′-ACCGCTGTGTGTGTACAGGC-3′ yielding a 169 bp piece, for exon 4 forward primer 5′-AGCTTCTCAACGGCTCTGC-3′ reverse primer 5′-ACAGACTCTCCA CGAGGTCG-3′ yielding a 207 bp piece, and for exon 6 forward primer 5′-CCTTCTCCCCACACTGCTAGG-3′ reverse primer 5′-GCAAGCATCCCCATCTCC AC-3′ yielding a 550 bp piece. Amplification conditions for exons 1 and 4 were 40 cycles consisting of a denaturing step at 95° C., an annealing step at 60° C. and an extension step at 72° C. for 45 seconds each step with 1.5 mM MgCl₂. Exon 6 differed both in the annealing temperature which was 65° C. and MgCl₂ concentration which was raised to 2 mM. All PCR's utilized Taq Polymerase (Life Technologies, Grand Island, N.Y.), PCR buffer of 20 mM Tris-HCl (pH 8.4) and 50 mM KCl and were carried out using a Perkin Elmer DNA Thermal Cycler 480 (PE Applied Biosystems, Foster City, Calif.). The products were run on 1.5% agarose gels and treated as a southern hybridization following the above protocol to confirm specificity of the product.

### RT-PCR of Cell Lines

RNA was isolated from each cell line using RNAzol B (Tel-Test, Inc., Friendswood, Tex.) following the manufacturer's protocol. Briefly, cells were rinsed 2 times in ice-cold PBS, 0.2 ml of RNAzol B was added per 10⁶ cells, followed by 0.2 ml chloroform per 2 ml of lysate, after which the mixture was shaken and incubated on ice, followed by centrifugation at 12,000×g at 4° C. RNA was precipitated by adding an equal volume of isopropanol to the aqueous phase and centrifuged at 12,000×g at 4° C.

Reverse transcription was carried out on 2 μg of total RNA from DU-145, PC-3, LNCaP, and TSU-pr1 cell lines and 1 μg of total RNA from the A875 cell line for 15 minutes at 42° C. using 2.5 units reverse transcriptase (Life Technologies, Grand Island, N.Y.) per RNA sample in 50 mM Tris-HCl (pH 8.3), 75 mM KCl, 10 mM DTT, 3 mM MgCl₂. The resulting reverse transcription reaction was subjected to PCR amplification using primers adapted from Schenone et al. (1996). These primers are forward primer 5′-AGCCCCCAATTCAGTCCGCAAA-3′ and reverse primer 5′-CAGCAGCCAGGATGGAGCAATAG-3′ which amplifies a 847 bp piece. Amplification was carried out through 45 cycles of denaturation at 95° C. for 60 seconds, followed by annealing-extention at 60° C. for 45 seconds in 20 mM Tris-HCl (pH 8.4), 50 mM KCl, 1.5 mM MgCl₂. The resulting amplification reaction of the prostate tumor cell lines were precipitated by addition of 3M sodium acetate and 100% isopropanol at −20° C. overnight. The precipitates were then electrophoresed on a 1% agarose gel with a 100-fold dilution of the A875 cell line amplification reaction used as a positive control. Southern hybridization, following the above protocol, was then carried out to confirm specificity of the product.

### RNase Protection Assay

To create the riboprobe, pMVC5A vector, which contains 1507 bp of the p75^{NTR} cDNA, was digested with both Sphl, which cuts at base 208, and Pvull, which made a blunt cut at base 943. The resultant fragment was excised from low-melting agarose (Sigma Chemical Co., St. Louis, Mo.) and was ligated into the Sphl and Smal site of the pGEM-4Z (Promega Corp., Madison, Wis.) vector. The cloned vector was then digested with Avail in order to create a 388 base riboprobe. For an internal control a GAPDH vector (gift of the Chrysogelos Lab, Lombardi Cancer Center) was used that yields a 110 base piece when cut with BamHI. Both riboprobes were created using T7 in vitro transcription (Ambion Inc., Austin, Tex.) using [α³²p] UTP (3000 Ci/mmol; Amersham Life Sciences, Inc., Arlington Heights, Ill.) for p75^{NTR} and [α³²P] UTP (800 Ci/mmol; Amersham Life Sciences, Inc., Arlington Heights, Ill.) with 100-fold cold UTP for GAPDH and A875 p75^{NTR} riboprobe formation. The in vitro transcription was incubated at 37° C. for 1 hour. Template DNA was removed with RNase-free DNase I at 37° C. for 15 minutes. RNase Protection was done using RPA II (Ambion Inc., Austin, Tex.) Ribonuclease Protection Assay Kit according to the manufacturer's protocol. Briefly, radiolabeled riboprobe was mixed with RNA, precipitated with 5M ammonium acetate and 2 volumes of ethanol, resuspended in hybridization buffer (80% deionized formamide/100 mM sodium citrate pH 6.4, 300 mM sodium acetate pH 6.4, 1 mM EDTA), denatured at 95° C. for 4 minutes, and hybridized at 45° C. overnight. Unbound RNA was then digested with RNase A/RNase T1, then precipitated with RNase Inactivation/Precipitation Mixture supplied with the kit. The protected fragments were then resuspended in gel loading buffer (95% formamide, 0.025% xylene cyanol, 0.025% bromophenol blue, 0.5 mM EDTA, 0.025% SDS), denatured at 95° C. for 4 minutes, electrophoresed on a 5% acrylamide/8M urea gel, exposed to Hyperfilm MP (Amersham Life Sciences, Inc., Arlington Heights, Ill.) autoradiography film and developed in a 100Plus Automatic X-ray Film Processor (All-Pro Imaging Corp., Hicksville, N.Y.).

### Transient Transfection

DU-145, PC-3 and TSU-pr1 cell lines were grown in 6-well plates (Corning, Corning, N.Y.) until approximately 60-70% confluent. 5 μl of lipofectamine (Life Technologies, Grand Island, N.Y.) was added to either 5 μg pCMV5A (gift of Barbara Hempstead) vector, which contains the first 1507 bases of the p75^{NTR} cDNA, or 9 μg pMVE1 (gift of Moses Chao) vector, which contains the full length p75^{NTR} cDNA of 3386 bases, and allowed to form complexes for 30-45 minutes. The cells were washed once in serum-free DMEM and then overlaid with the lipofectamine/vector complex and incubated for 6 hours at 37° C. in 10% CO₂/95% air. After 6 hours the solution containing the lipofectamine/vector complex was removed and replaced with DMEM containing 5% FBS and 10 ng/ml 2.5S Nerve Growth Factor (Becton Dickinson, Franklin Lakes, N.J.) and allowed to recover 24-48 hours.

### PCR of Transiently Transfected Cells

DNA was obtained from the transiently transfected cells with the Wizard Genomic DNA Purification System (Promega, Madison, Wis.) according to the manufacturer's directions. Briefly, cells were harvested, pelleted at 16,000×g, lysed in Nuclei Lysis Solution, treated with RNase Solution, proteins precipitated with Protein Precipitation Solution, centrifuged at 16,000×g, the supernatant mixed with isopropanol at room temperature, centrifuged at 16,000×g, washed with 70% ethanol, and rehydrated in DNA Rehydration Solution. The genomic DNA was then subjected to 35 cycles of denaturation at 95° C., annealed at 65° C. and extension at 72° C. for 45 seconds each in 20 mM Tris-HCl (pH 8.4), 50 mM KCl and 1.5 mM MgCl₂, with the primers used in RT-PCR above to distinguish the inserted cDNA from the gene. The samples were then run on a 1% agarose gel stained with ethidium bromide.

The loss of tumor suppressor gene function contributes to the transformation of human prostate epithelial cells to a malignant pathology. One such tumor suppressor gene has been mapped to the vicinity of 17q21, which happens to be coincident with the human p75^{NTR} gene locus. The neurotrophin receptor, p75^{NTR}, is expressed in normal human prostate epithelial cells, and exhibits an inverse association of p75^{NTR} expression with the malignant progression of the prostate, consistent with a pathological role of the p75^{NTR} as a putative tumor suppressor. Utilizing stable transfectants of the TSU-pr1 human prostate tumor cell line that exhibit a rank order (dose-dependent) increase in p75^{NTR} protein expression we investigated the effects of p75^{NTR} expression on the suppression of tumor cell growth. A rank order increase in the expression of p75^{NTR} protein in these tumor cells resulted in a significant increase in the percentage of cells that accumulated in G0/G1 and concurrently a significant decrease in the proportion of cells that accumulated in both S and G2-M phases of the cell cycle in vitro. When these prostate tumor cells were injected into the flanks of SCID mice, growth of the tumors was found to be inversely proportional with the level of p75^{NTR} expression. This dose-dependent effect of p75^{NTR} mediated suppression of tumor growth was associated with a dramatic decrease in tumor cell proliferation, as indicated by PCNA expression, and a modest increase in tumor cell apoptosis, as indicated by TUNEL, in vivo. These results provide formal identification of the p75^{NTR} as a new tumor suppressor in human prostate cancer.

The results discussed below show that the dose-dependent expression of p75^{NTR} induces GO/G1 cell cycle arrest in vitro; that dose-dependent expression of p75^{NTR} inhibits prostate tumor growth in an immunocompromised murine model, and that p75^{NTR} dependent inhibition of tumor growth is manifest as the decreased proliferation and increased apoptosis of the tumor cells in vivo.

### Increased p75^{NTR} Expression Increases Accumulation of Tumor Cells in the G0/G1 Phase of the Cell Cycle

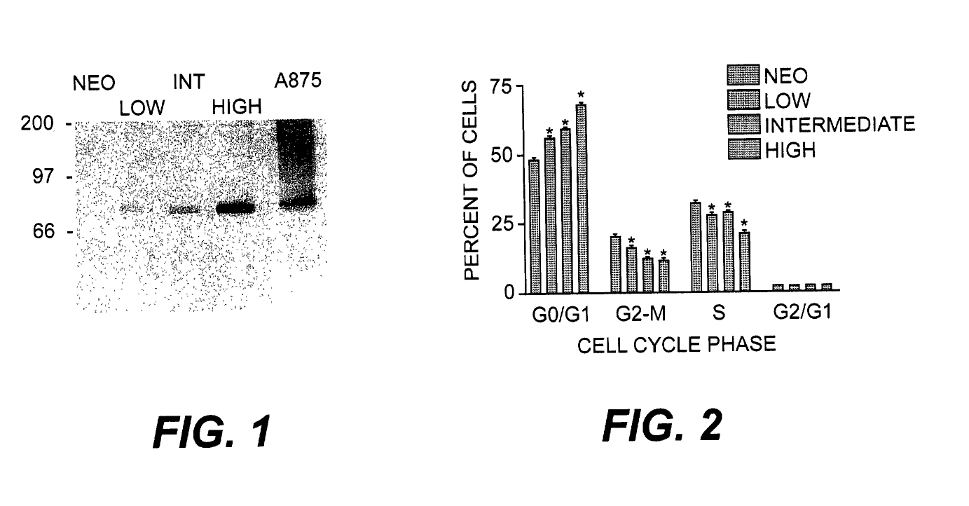

Representative clones of the TSU-pr1 human prostate tumor cell line that exhibit a graded (dose-dependent) increase in expression of the p75^{NTR} protein (FIG. 1) were used to determine the effects of p75^{NTR} expression on the cell cycle of these cells (FIG. 2). A rank order increase in p75^{NTR} expression was associated with a significant (p<0.000001) increase in the percentage of cells that accumulated in G0/G1 (FIG. 2). Approximately 48% of the cells in the neo control group were in G1/G1, which increased to 56% for the low p75^{NTR} expression cells, which further increased to 59% for the intermediate p75^{NTR} expression cells, while a maximum of 68% of the high p75^{NTR} expression cells were in G0/G1. Whereas increased p75^{NTR} expression was associated with an accumulation of cells into G0/G1, concurrently there was a significant (p<0.000001) decrease in the proportion of cells that accumulated in both G2-M and S phases of the cell cycle. Approximately 20% of the neo cells were in G2-M while 39% of these cells were in S phase. Approximately 16% of the low p75^{NTR} expression cells were in G2-M and 28% were in S phase. The intermediate p75^{NTR} expression cells exhibited 12% accumulation in G2-M, with approximately 28% of the cells in S phase, whereas the high p75^{NTR} expression cells exhibited the fewest proportion of cells in G2-M (11%) with 21% in S phase. Hence, a rank order increase in p75^{NTR} expression was associated with an accumulation of cells in G0/G1 and a reduction in the proportion of cells in both S and G2-M phases of the cell cycle, consistent with increased quiescence of these tumor cells.

### Increased p75^{NTR} Expression Decreases Tumor Volume in SCID Mice

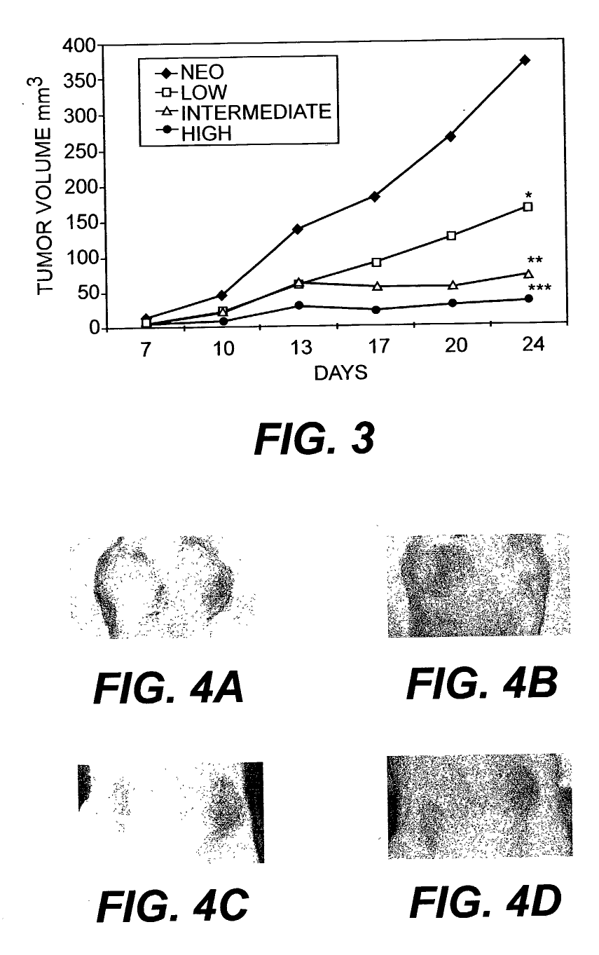

To investigate whether the observed effects of p75^{NTR} expression on the cell cycle in vitro would manifest as differences in the growth of prostate tumors in vivo, we employed a SCID mouse model of prostate tumor growth. Representative clones of the neo control, low p75^{NTR} , intermediate p75^{NTR} and high p75^{NTR} expression tumor cells (FIG. 1) were injected subcutaneously into the flanks of SCID mice. Prostate tumors formed by the neo TSU-pr1 cells exhibited the greatest rate of growth compared with tumors formed from any of the p75^{NTR} expressing TSU-pr1 cells. A rank order increase of p75^{NTR} expression (FIG. 1) in the tumor cells was associated with a decrease in the volume of tumors formed in SCID mice (FIG. 3). Compared to the neo control tumors (FIG. 4A) there was a significant reduction in the volume of low p75^{NTR} expression tumors (FIG. 4B) (p<0.05), intermediate p75^{NTR} expression tumors (FIG. 4C) (p<0.0005) and high p75^{NTR} expression tumors (FIG. 4D) (p<0.00005). Hence, p75^{NTR} protein expression appears to suppress in a dose-dependent manner the growth of tumors in SCID mice.

### Increased p75^{NTR} Expression Increases Apoptosis within the SCID mice Tumors

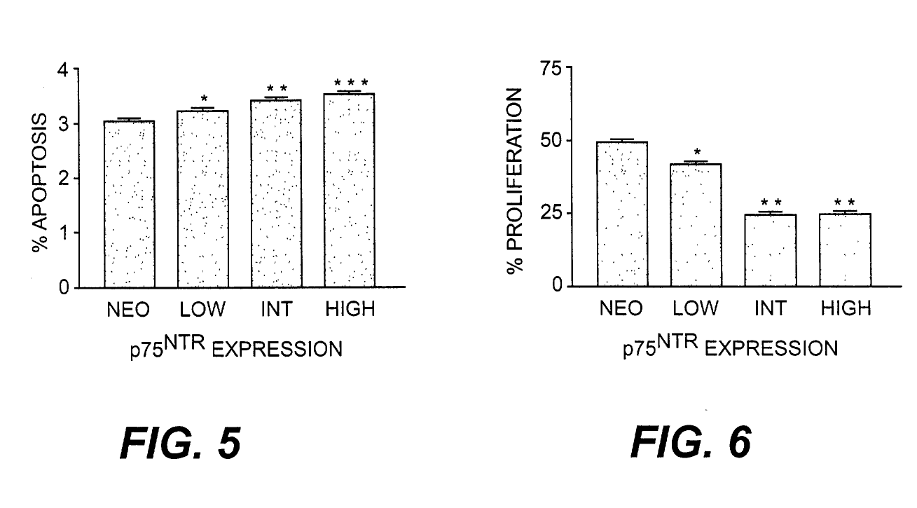

To investigate whether an increased proportion of cells undergoing apoptosis could account, in part, for the differences seen in the volume of tumors (FIGS. 3 & 4), serial sections of the neo, low, intermediate and high p75^{NTR} expression tumors were stained for apoptosis using the TUNEL technique. Approximately 3% of neo tumor cells were apoptotic, as indicated by the TUNEL technique (FIG. 5). The percentage of apoptotic cells increased to 3.2% in the low p75^{NTR} expression tumors (p<0.05), which increased further to 3.4% in the intermediate p75^{NTR} expression tumors (p<0.005), reaching a maximum of 3.6% (p<0.0005) of apoptotic cells in the high p75^{NTR} expression tumors (FIG. 5). Hence, the rank order increase of p75^{NTR} expression in the tumor cells (FIG. 1) was associated with a modest, but significant, increase in the proportion of apoptotic cells within the prostate tumors formed in SCID mice (FIG. 5).

### Increased p75^{NTR} Expression Decreases Proliferating Cells within the SCID Mice Tumors

Since differences in apoptosis may not fully account for the differences in decreased tumor volumes associated with increased p75^{NTR} protein expression, we investigated the association between p75^{NTR} expression and cell proliferation with the SCID mice tumors. The same tumors used in the apoptosis study (FIG. 5) were used to determine the proportion of cells that exhibited the proliferating cell nuclear antigen (PCNA) that has been shown to distinguish those cells committed to proliferate. The percentage of proliferating cells was calculated by dividing the number of nuclei stained positively for PCNA expression by the total number of counted nuclei. A rank order increase in p75^{NTR} expression in the tumor cells (FIG. 1) was associated with a dramatic decrease in the percentage of cells that were undergoing proliferation, as indicated by PCNA expression (FIG. 6). In the neo control, 50% of the cells exhibited PCNA expression, while 42% of the low p75^{NTR} expression tumors expressed PCNA (p<0.005), which decreases further to 26% in the intermediate p75^{NTR} expression tumors (p<0.000005) and 25% in the high p75^{NTR} expression tumors (p<0.000005). Hence, the rank order increase of p75^{NTR} expression in the tumor cells (FIG. 1) was associated with a highly significant decrease in the proportion of cells committed to proliferation, as indicated by PCNA expression (FIG. 6).

Proteins involved in the formation of cancers have been functionally classified into two basic types: the products of oncogenes and tumor suppressors. Tumor suppressors such as p53 and BRCA1, are characterized by either a loss of expression or function, which removes growth inhibitory signals, thereby facilitating tumorigenesis. The pathologic loss of tumor suppressor proteins such as the transcription factor AP-2, has been demonstrated during progression from normal breast tissue to invasive carcinoma (19), and the loss of gp200-MR6 expression with increasing malignancy in colorectal carcinoma (20). Significantly, expression of p75^{NTR} is also progressively lost during malignant transformation of prostate epithelial cells in man. Studies utilizing immunoblot techniques (9), immunofluorescence (10), immunohistochemistry (12) and Scatchard plot analysis (12) have all confirmed reduced expression of p75^{NTR} protein with the malignant transformation of the human prostate. Loss of expression of p75^{NTR} is also correlated with Gleason's score of pathological prostate tissues (13). Whereas pre-malignant epithelial cells of normal and PIN pathology retain full expression of the p75^{NTR} protein, well differentiated prostate adenocarcinoma tissues show a large decline in the proportion of epithelial cells that express the p75^{NTR} protein (13). Moderate and poorly differentiated prostate adenocarcinoma tissues exhibit an even larger proportion of epithelial cells that have lost expression of the p75^{NTR} protein (13). This receptor is also absent from four human epithelial tumor cell lines derived from prostate metastases (9), indicating an inverse association of p75^{NTR} expression with the malignant progression of the human prostate, as has been demonstrated during pathological progression of several well characterized tumor suppressors.

Constitutive expression of p75^{NTR} protein in stably transfected TSU-pr1 human prostate tumor cells in vitro resulted in an increase in the percentage of cells in the G0/G1 phase of the cell cycle. This effect was in direct proportion with p75^{NTR} expression, whereby a rank order increase in p75^{NTR} expression was associated with increased accumulation of tumor cells in the G0/G1 phase of the cell cycle, and concomitantly reduced accumulation of tumor cells in the S phase and G2-M phases of the cell cycle. This effect of p75^{NTR} expression is consistent with other tumor suppressors such as p53 (21; 22), p73 (23), Smad 4/DPC 4 (24), and p21 (25) which have been shown to cause G0/G1 cell cycle arrest. Expression of p75^{NTR} protein by transient transfection in the same TSU-pr1 human prostate tumor cells in vitro was also shown to induce an increase in the rate of apoptosis (16). Hence, it seems clear that p75^{NTR} functions to arrest prostate tumor cells in G0/G1 , and also to induce some tumor cells to undergo apoptosis. A comparable dual function following re-introduction of tumor suppressors to arrest the cell cycle in G0/G1 and enhance apoptosis has similarly been demonstrated for p53 (22), p73 (23), and Smad 4/DPC 4 (24).

Most significantly, p75^{NTR} mediated cell cycle arrest of tumor cells in vitro was associated with a concomitant dose-dependent inhibition of tumor growth in vivo. This result provides formal characterization of p75^{NTR} as a tumor suppressor of prostate tumor growth. Since growth is the net result of cell proliferation minus cell death, we examined the proportion of cells undergoing proliferation, as determined by PCNA expression, and the proportion of cells undergoing apoptosis, as determined by the TUNEL assay. Clearly, a dose-dependent increase in p75^{NTR} mediated tumor suppression was associated with a dramatic decrease in tumor cell proliferation and a modest increase in tumor cell apoptosis in vivo. Interestingly, several receptor proteins exhibit overlapping sequence identity with p75^{NTR} , These homologous cell surface receptors include the tumor necrosis factor receptors (p75^{TNFR}, p55^{TNFR}), Fas, and the TRAIL receptors (DR3, DR4, DR5). At least three of these receptors (p75^{NTR} , p55^{TNFR}, Fas) share similar sequence motifs of three to four repeats of defined elongated structure (26) which have been designated “death domains” based upon their ability to induce apoptosis (27). Based upon the ability of Fas and the TNF receptors to induce apoptosis in vitro, it has generally been assumed that these receptors may be putative tumor suppressors. However, it is believed that the results included in the subject application for the first time formally characterize a member of the TNF receptor-super family, the p75^{NTR} , as a tumor suppressor in vivo. It has long been known that Fas and TNF receptors can induce apoptosis, but both receptors require addition of ligand for their action. The mere presence of these receptors appears insufficient to induce apoptosis, although recently KILLER/DR5 was identified as a possible mediator in p53-dependent apoptosis in head and neck cancer (28). In contrast, p75^{NTR}, outside of the central nervous system, has been shown to induce apoptosis through the withdrawal of ligand (15, 16, 29, 30, 31) as well as induce G0/G1 cell cycle arrest and reduce proliferation of tumor cells.

In conclusion, we have formally identified p75^{NTR} as a tumor suppressor within the human prostate. The locus of the p75^{NTR} gene as closely distal to 17q21 (8) is consistent with a high frequency loss of heterozygosity in prostate cancer in the vicinity of 17q21 (4, 6), and its association with a putative prostate tumor suppressor gene in the vicinity of 17q21 (4, 5, 6). Moreover, the progressive loss of p75^{NTR} protein expression associated with the malignant progression of the human prostate (13, 12, 9, 16) is consistent with a pathological role of p75^{NTR} as a tumor suppressor. The loss of expression of the p75^{NTR} tumor suppressor within the malignant prostate would appear to reduce G0/G1 cell cycle arrest as well as reduce apoptosis and increase proliferation of tumor cells, thereby contributing to the growth of prostate tumors in the absence of the p75^{NTR} tumor suppressor.

### Cell Cycle Modification of Tumor Cells by stable Expression of the p75^{NTR} In Vitro

To evaluate the re-gain of function of the putative p75^{NTR} suppressor gene in TSU-pr1 and DU-145 human prostate tumor cell lines we utilized stable expression of p75^{NTR} The dose-dependent level of p75^{NTR} expression is evaluated in relation to cell proliferation and changes in the cell cycle kinetics of the transfectants. These studies demonstrate whether p75^{NTR} expression reduces entry into S phase and/or increases apoptotic DNA fragmentation as a corollary of growth inhibition.

### Inhibition of Tumor Growth following Induced Expression of the p75^{NTR} In Vivo

The results provided herein allow the evaluation of the relationship between p75^{NTR} dependent suppression of tumor growth in SCID mice via either the induction of programmed cell death and/or reduced cell proliferation in the tumors. Half of the tumors are analyzed for p75^{NTR} gene expression while the other half are analyzed for the proportion of cells exhibiting immunohistochemical co-localization of p75^{NTR} protein with induction of programmed cell death (TUNEL assay) and/or cell proliferation determined by proliferating cell nuclear antigen (PCNA) assay. These studies establish a mechanistic relationship between the tumor suppressive effects of p75^{NTR} expression and the rate of programmed cell death and the rate of proliferation of the tumor cells, thereby formally demonstrating that the p75^{NTR} is a tumor suppressor

### Tumor Growth, p75^{NTR} Gene Expression, Immunohistochemistry, TUNEL and PCNA Analysis

All SCID mice procedures are performed in the Animal Resource Facility by Tumor Biology Program staff from the Lombardi Cancer Center at Georgetown University. p75^{NTR} expressing tumor cells (1×10⁶) as described above, are resuspended 1:1 in Matrigel™ (final vol. Of 150 μ1) and injected subcutaneously into the abdomen of SCID mice. Tumor size is determined with calipers every second day. Upon removal, tumors are weighted, bisected and one half analyzed for p75^{NTR} gene expression by Northern blot using the cDNA probe excised from the p75^{NTR} expression vector, according to our published protocols (26).

The remaining half of the tumors are snap frozen or fixed for sectioning and stained with an anti-human p75^{NTR} monoclonal antibody (Boehringer Mannheim), or control mouse IgG (Cappel) followed by a standard biotinylated streptavidin-alkaline phosphatasekit (Zymed). This produces a red streptavidin-alkaline phosphatase immunoreactivity in the plasma membrane and cytoplasm of the p75^{NTR} positive cells, as we have previously demonstrated (7). Subsequently, each of the sections stained by p75^{NTR} immunohistochemistry alternatively be stained either by the deoxynucleotide transferase mediated dUTP biotin nick end labeling (TUNEL) assay (27) according to manufactures specifications, or for proliferating cell nuclear antigen (PCNA) localization (28), according to standard protocols. Subsequently, at least 1000 adjacent cells are scored for PCNA immunoreactivity (28). The proliferation index (PI) of PCNA immunostained material is calculated using the formula PI (%)=A/B×100 where A=the total number of PCNA labeled nuclei, and B=the total number of nuclei. Since, the p75^{NTR} protein localizes as diffuse red reaction product to the cytoplasm and both the TUNEL and PCNA assays localize to the nucleus as discrete black reaction product, co-localization of p75^{NTR} protein with either TUNEL or PCNA is readily distinguishable. Subsequently, the proportion of cells that stain individually with each of these techniques and/or co-stain with p75^{NTR} protein and either TUNEL or PCNA is quantified on an image analysis system (Omnicon 3600, Imigin Products Inc., Chantilly, Va.) in the Lombardi Cancer Research Center at Georgetown University.

The invention is based on the unexpected identification of p75^{NTR} as a tumor suppressor of prostate cancer. By formally identifying the p75^{NTR} protein as a tumor suppressor the invention provides a mechanistic link between the pathologic loss of p75^{NTR} protein expression and its role in the progression of prostate cancer. Formal identification of the p75^{NTR} protein as a tumor suppressor has several implications with regard tot he clinical potential of the present inventon. 1) The p75^{NTR} suppressor may be developed as a diagnostic and prognostic marker of prostate tumor progression, much in the same way that estrogen receptor (ER) negative pathologies are used in the assessment of breast cancers, and 2) identification of the p75^{NTR} as a tumor suppressor may form the basis of gene therapy studies for inhibition of human prostate cancer.

The p75^{NTR} gene contains 6 exons (Chao et al., 1986, Sehgal et al., 1988) which map in the region of q21-22 on chromosome 17 (Huebner et al., 1986, Rettig et al., 1986, Van Tuinen et al., 1987). Interestingly, loss of the q arm of chromosome 17 has been associated with some prostate cancers (Lalle et al., 1994, Gao et al., 1995a, Gao et al., 1995b). Hence, using a restriction map of the p75^{NTR} gene (Chao et al., 1986, Sehgal et al., 1988), we investigated the mechanism(s) by which loss of p75^{NTR} expression occurs in four naturally occurring human prostate tumor cell lines. We demonstrate by Southern blot analysis that the gene is present in all four tumor cell lines with no deletions within the gene, RT-PCR and RNase protection shows that transcription of mRNA occurs within these cells, and finally using transient transfections we show that p75^{NTR} protein expression is due, at least in part, to decreased mRNA stability.

### Southern Hybridization and PCR

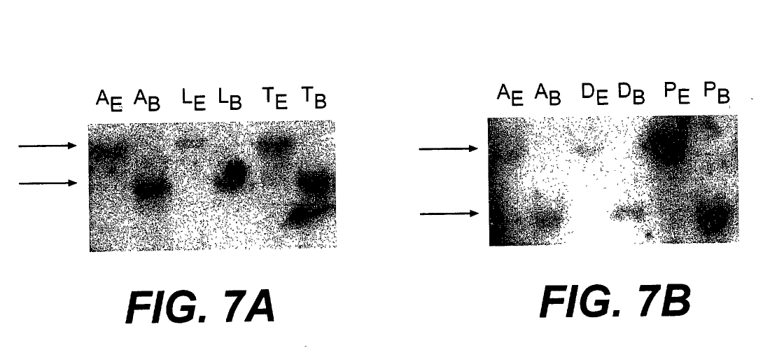

To examine whether the p75^{NTR} gene contains deletions, or is itself deleted, Southern hybridization was carried out. Using the restriction map in Sehgal et al. (1988), EcoRI and BamHI were used to digest genomic DNA from the four human prostatic epithelial tumor cell lines DU-145, PC-3, LNCaP, and TSU-pr1 , and the human melanoma A875 cell line as a positive control. Hybridization of a radiolabeled probe created from the full-length cDNA showed the same digestion pattern (FIG. 7 A and B) in all four of the tumor cell lines when compared to the A875 cell line. The band at approximately 10 kb in the EcoRI lanes (represented by subscript E) represents the 3′ portion of the gene that includes exons 3 through 6. The band at approximately 4 kb in the BamHl lanes (represented by subscript B) are composed of fragments that contain all six of the exons. The Southern hybridization also shows that the 3′ end of the gene is present in all tumor cell lines.

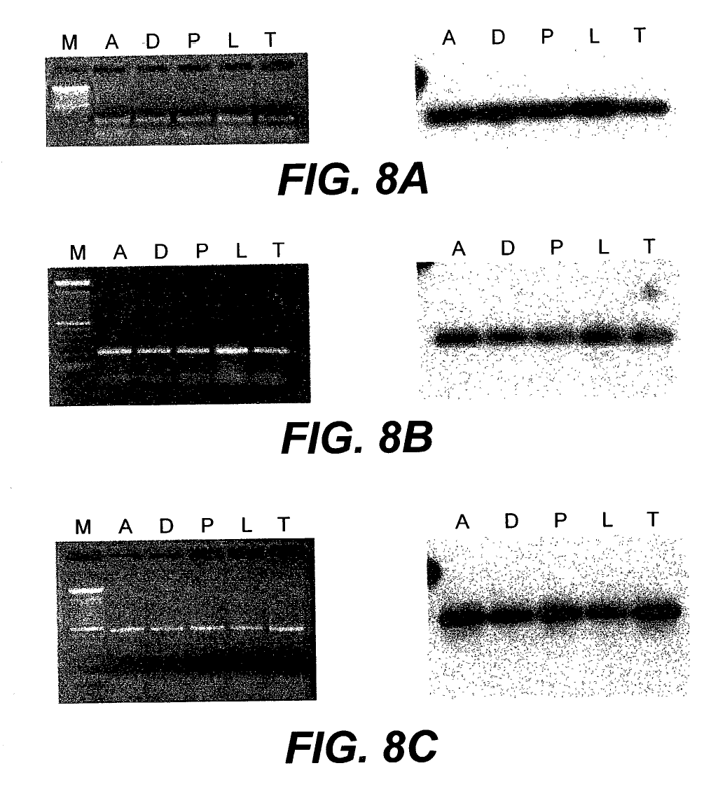

To examine if the 5′ fragment of the gene is present, Polymerase Chain Reaction (PCR) amplification of specific exons was undertaken. This not only allows examination of the exons in the 5′ fragment of the gene, but also serves to re-confirm the Southern analysis. Exon 1 (FIG. 8A), exon 4 (FIG. 8B) and exon 6 (FIG. 8C) were amplified using the primers listed above in the Materials and Methods section, run on 1% agarose gels and hybridized against the p75^{NTR} cDNA probe to ensure that the amplified fragment was specific for the p75^{NTR} gene (right panels in FIG. 8). Exon 2 was not amplified because the exon itself is very small, and the amplified fragment would be too small to visualize on an agarose gel. As shown, all three exons are present in all four of the tumor cell lines and the A875 positive control.

Taken together, both the Southern analysis and PCR amplification indicate that the gene was not lost during malignant progression of the cell lines. It also shows that there are no gross deletions within the gene itself, and that all four prostate tumor cell lines and the positive control A875 melanoma cell line are identical with respect to Southern analysis and PCR amplification of the exons.

### RT-PCR and RNase Protection

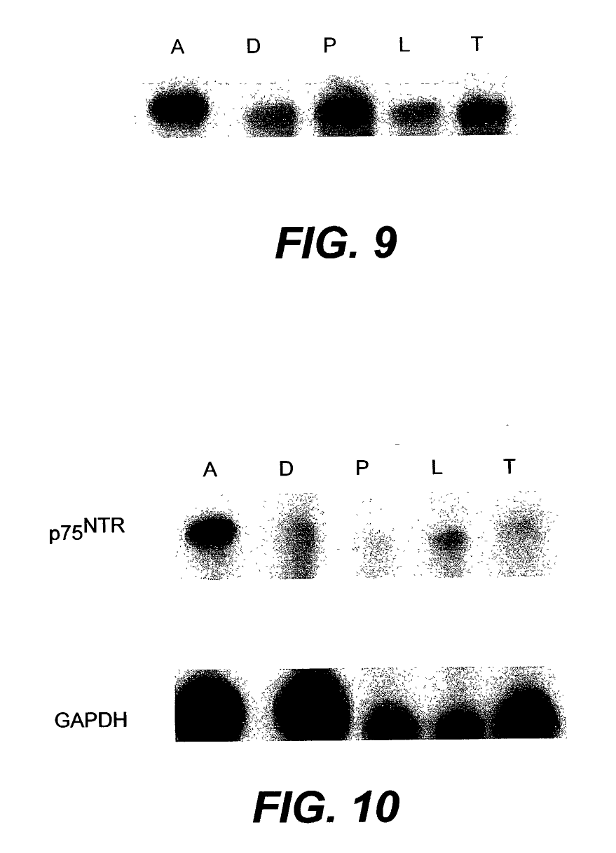

Since Southern analysis and PCR amplification demonstrated that the p75^{NTR} gene is present, we examined the transcription of the p75^{NTR} gene. Reverse Transcription-Polymerase Chain Reaction (RT-PCR) using primers adapted from Schenone et al. (1996), as described in above. The primers amplify the mRNA from base 52 to base 897 yielding a 847 bp piece. As seen in FIG. 9, mRNA is present, albeit at an extremely low level. It is important to note that the A875 lane is a 100-fold dilution of the PCR sample, while the lanes of the prostate tumor lines contain the entire sample amount. Also, the bands were not seen in the ethidium bromide stained gel, but only upon specific hybridization with the radiolabeled probe.

To re-confirm the RT-PCR results, an RNase Protection Assay (FIG. 10) was performed. Again, mRNA was present in all four tumor cell lines at a low level as well as in the positive control A875 cells at a much higher level. In this instance, 100-fold cold UTP was used to create the GAPDH probe used in all the cell lines and in the p75^{NTR} probe used for the A875 cell line.

Both sets of data (RT-PCR and RNase protection analysis) clearly indicate that the transcription machinery is present within these human prostate tumor cells, since a small amount of mRNA is present.

### Transient Transfections

In order to determine whether mRNA stability may play a role in the low abundance of the transcript in the prostate tumor cell lines, transient transfections using two versions of p75^{NTR} cDNAs were used. pCMV5A contains the first 1507 bases of p75^{NTR}, containing the 5′ untranslated region (UTR), the full open reading frame (ORF), and only about 200 bases of the 3′UTR, while pMVE1 contains the full-length cDNA, including the 2 kb 3′ UTR. Both constructs are under identical CMV promotion. Equimolar concentrations of each vector were transfected into DU-145, PC-3 and TSU-pr1 cells, and the cells were allowed to recover in the presence of 10 ng/ml NGF prior to isolation of protein and DNA.

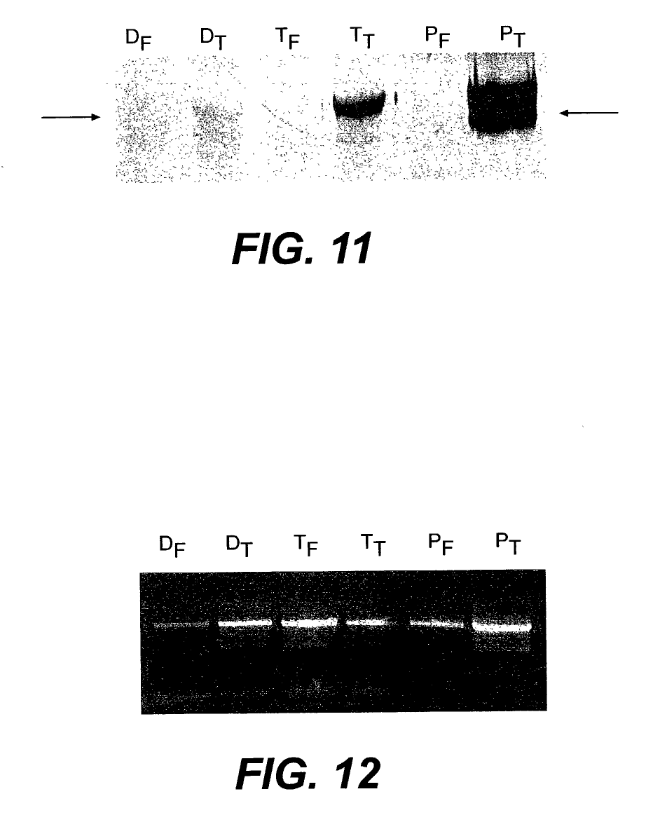

Upon Western blot analysis (FIG. 11), the truncated vector pCMV5A (denoted by subscript T) displayed high p75^{NTR} protein expression. In contrast, the full length vector pMVE1 (denoted by subscript F) showed either low or non-existent p75^{NTR} protein expression. The blot shown was representative of three independent experiments.

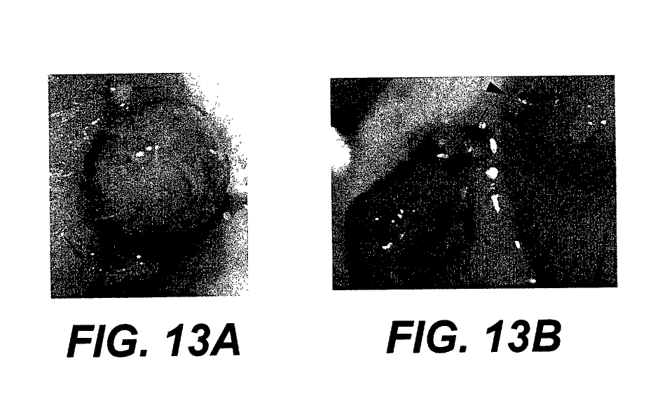

To rule out the possibility that pMVE1 was not incorporated into the cells, PCR was performed on the genomic DNA isolated from both vector constructs transfected into cells. The primers used were the same used in the RT-PCR. This was so we could distinguish the transfected p75^{NTR} cDNA from the endogenous gene, since the primers would span several exons. As seen in FIG. 12, both vector constructs were incorporated into the cell lines.

Expression of the p75^{NTR} protein is progressively lost in pathologic human prostate tissues. Both normal prostate tissue and prostatic intraepithelial neoplastic (PIN) tissue exhibit intense staining of p75^{NTR} in all epithelial cells, whereas in the neoplastic prostate a proportion of epithelial cells exhibit loss of p75^{NTR} expression (Perez et al., 1997). Significantly, the proportion of epithelial cells that have retained p75^{NTR} expression in the organ confined pathological prostate is inversely associated with increasing Gleasons score and pre-operative serum PSA concentrations (Perez et al., 1997). Hence, loss of p75^{NTR} expression may be indicative of the early stages of neoplastic transformation of the prostate. In addition, Western blot of the human prostate epithelial cell lines TSU-pr1, DU-145, PC-3 and LNCaP derived from metastases showed a complete absence of p75^{NTR} expression (Pflug et al., 1992). This was further confirmed by Scatchard plot analysis which showed an absence of p75^{NTR} on TSU-pr1 prostate tumor cells (Pflug et al., 1995). Hence, complete expression of p75^{NTR} in the normal prostate, partial loss of p75^{NTR} expression in organ confined adenocarcinoma tissues (Pflug et al., 1992, Pflug et al., 1995, Perez et al., 1997, Dionne et al., 1998), and complete loss of p75^{NTR} expression in four prostate epithelial tumor cell lines derived from metastases (Pflug et al., 1992) shows progressive loss of p75^{NTR} expression with the malignant progression of the human prostate. Many prostate tumor cell lines have been created in the laboratory by various transformation methods. However, there are only four naturally occurring human prostate epithelial cell lines (TSU-pr1, DU-145, PC-3, LNCaP) that have been isolated from prostate cancer patients (Pflug et al., 1992). It seems highly significant that all four of these naturally occurring human prostate tumor cell lines have coincidentally lost expression of the p75^{NTR} protein (Pflug et al., 1992). Hence, we investigated the mechanism by which all four human prostate tumor cell lines have lost expression of the p75^{NTR} protein, and whether the same or different mechanisms of lost expression occurs for each of the four cell lines.

Since the p75^{NTR} gene is located on chromosome 17 in the region q21-22 (Huebner et al, 1986, Rettig et al., 1986, VanTuinen et al., 1987), and that loss of regions of the q arm of chromosome 17 has been associated with some prostate cancers (Lalle et al.,1994, Gao et al., 1995a, Gao et al., 1995b), we initially investigated whether the loss of expression of p75^{NTR} may be due to the partial or complete deletion of the gene. Southern blot analysis showed an identical endonuclease restriction pattern between all four prostate tumor cell lines that have been shown not to express the p75^{NTR} protein (Pflug et al., 1992), and the unrelated A875 human melanoma cell line that is known to overexpress the p75^{NTR} protein (Fabricant et al., 1977, Ross et al., 1984). Since it is extremely unlikely that an identical partial deletion would occur in all four prostate tumor cell lines, and since the A875 human melanoma cell line had an identical Southern blot restriction pattern to these four prostate cell lines, and yet the A875 human melanoma overexpresses the p75^{NTR} protein (Fabricant et al., 1977, Ross et al., 1984), it seems clear that the p75^{NTR} gene has remained intact within these four prostate tumor cell lines. This conclusion was further supported by the PCR of exons 1, 4 and 6 from genomic DNA extracted from each of the four prostate cell lines. Examination of the promoter region of the p75^{NTR} gene (Sehgal et al.,1988) shows that although the promoter does not contain either a CMT or TATA box, there are several GGGCGG sequences (considered a GC rich region). This sequence is similar to the sequence that is recognized by the transcription factor Sp1 (Dynan and Tijan, 1983). This type of promoter is seen in many constitutively expressed housekeeping genes such as hypoxanthine phosphoribosyltransferase (Melton et al., 1984), 3-hydroxy-3-methyl-glutaryl-coenzyme A reductase (Reynolds et al., 1984), adenosine deaminase (Valerio et al., 1985), and metaxin (Collins and Bornstein, 1996) as well as receptors including the neuronal nicotinic receptor a7 subunit (Carrasco-Serrano et al., 1998), the α5 subunit (Campos-Caro et al., 1999) and the p75^{NTR} gene of the rat (Poukka et al., 1996). There are also reports that in addition to the SP1 binding proteins, other proteins are needed to complex with SP1 which will affect transcription rate (Chiaramello et al., 1995; Poukka et al., 1996; Wang et al., 1999). Since the p75^{NTR} gene is present in all four prostate tumor cell lines, and the characteristics of the promoter region is consistent with the constitutive regulation of transcription, we investigated whether transcription was occurring in the prostate tumor cell lines. RT-PCR analysis and RNase protection assay both confirmed that mRNA is being transcribed, although the amount in the prostate cell lines was approximately 100-fold less than that of the A875 human melanoma cell line. The RT-PCR results are also significant since the resultant cDNA product was encoded by exons 1, 2 and 3. These exons identified by RT-PCR in conjunction with the PCR of exons 1, 4 and 6 from genomic DNA, and the EcoRI Southern blot of exons 3 through 6, show that all the exons that encode the p75^{NTR} ORF are intact in these prostate tumor cell lines. Hence, the p75^{NTR} gene appears intact and is being transcribed in all four tumor cell lines in a similar manner.

In order to address whether elements of the 3′ UTR may affect stability of the mRNA produced by the prostate tumor cell lines, we utilized two constructs of the p75^{NTR} cDNA that only differ in the 3′ UTR. The pCMV5A construct contains the 5′ UTR, ORF, and a very short 3′ UTR of 100-200 bases, whereas the pMVE1 construct contains the 5′ UTR, ORF and the complete 3′ UTR of 2 kb. Both constructs are under identical CMV promotion, and therefore they should both be expressed at levels easily detected by Western blot analysis. If the loss of p75^{NTR} expression was due solely to a decrease in the transcription rate, then both vector constructs, when used in transient transfection, would have expressed appreciable levels of p75^{NTR} protein. Only the pCMV5A vector, which lacks most of the 3′ UTR, expressed an appreciable level of p75^{NTR} protein. The pMVE1 vector, which contains the full 3′ UTR of 2 kb did not express p75^{NTR} protein at any appreciable level. Since the p75^{NTR} contains a promoter that has been implicated in constutively active gene expression, and there was a significant difference in the expression levels between the two vector constructs, there must be another explanation for the loss of p75^{NTR} expression. Indeed, the 3′ UTR has been shown to contribute an important role in protein expression through mRNA stabilization. The mda-7 gene contains AU-rich elements which contribute to the rapid turnover rate of the mRNA (Madireddi et al., 2000), while many other mRNAs contain structural motifs that bind cytosolic proteins to stabilize mRNA, such as transferrin receptor (Müllner and Kühn, 1988), mammalian ribonucleotide reductase component R2 (Amara et al, 1995; Amara et al., 1996a), Hyaluronan receptor RHAMM (Amara et al., 1996b), glucose transporter (McGowan et al., 1997), H-ferritin (Ai and Chau, 1999), and chicken elastin (Hew et al., 1999). In this context, the Western blot data, from the transient transfections supports the idea that mRNA instability, mediated by an element(s) of the 3′ UTR, is playing a role in the loss of p75^{NTR} protein expression. In conclusion, in all four naturally occurring human prostate tumor cell lines, it appears that the p75^{NTR} gene has remained intact, that transcription of the gene occurs, but that a low abundance of mRNA, resulting at least in part from decreased mRNA stability, results in a loss of p75^{NTR} protein expression.

### The Dual Role of the p75^{NTR} as a Metastasis Suppressor of Prostate Tumor Cells

NGF, the predominant ligand for p75^{NTR} in the human prostate, appears to promote metastasis of prostate cancer via perineural invasion along perineural spaces that exhibit intense NGF immunoreactivity, as supported by in vitro Boyden chamber assays of chemomigration. To examine the effects of NGF on tumor growth and metastasis in vivo, 0-100 ng/ml of NGF was injected every two days into the site of tumor cell growth. NGF did not significantly affect the overall growth of the tumors. However, NGF stimulated the formation of contiguous and non-contiguous tumors in a dose-dependent manner. Metastastic tumor spread was described as contiguous if they formed an outgrowth from the primary tumor but remained attached (FIG. 13, arrows), or non-contiguous if they occurred at a distant site from the primary tumor (FIG. 13, arrow heads).

Table 1 shows the dose-dependent effects of NGF and increasing p75^{NTR} expression in TSU-pr1 and PC-3 tumor cells on the metastatic spread of tumors from the primary site after 25 days.

TABLE 1
Dose-Dependent Effects of NGF and p75^{NTR} Expression on
Tumor Metastasis

| | 0 ng/ml NGF | | 10 ng/ml NGF | | 100 ng/ml NGF | |
|---|---|---|---|---|---|---|
| | | Non- | | Non- | | Non- |
| | Con- | Con- | Con- | Con- | Con- | Con- |
| | tiguous | tiguous | tiguous | tiguous | tiguous | tiguous |
| TSU | 1*# | 0.2*# | 1.8*# | 1.4*# | 2.2*# | 3.6*# |
| pr1 | | | | | | |
| Neo | | | | | | |
| TSU- | 1*# | 0.4*# | 1.8*# | 1*# | 1.8*# | 1.6*# |
| pr1 | | | | | | |
| Low | | | | | | |
| TSU- | 1*# | 04*# | 1.8*# | 1*# | 0.8*# | 1*# |
| pr1 | | | | | | |
| Int. | | | | | | |
| TSU- | 0*# | 0*# | 0*# | 0*# | 0.2*# | 0*# |
| pr1 | | | | | | |
| High | | | | | | |
| PC-3 | 1*Ψ | 0.2*# | 1.6*Ψ | 1*# | 1.8*Ψ | 2*# |
| Neo | | | | | | |
| PC-3 | 0*Ψ | 0*# | 0*Ψ | 0*# | 0*Ψ | 0*# |
| Low | | | | | | |
| PC-3 | 0*Ψ | 0*# | 0*Ψ | 0*# | 0*Ψ | 0*# |
| High | | | | | | |

Table 1 shows NGF promotes the dose-dependent spread (contiguous) and metastasis (non-contiguous) of tumors, while increasing p75^{NTR} expression (Neo to High) suppresses the spread and metastasis of tumors.

All numbers are means per mouse. *p<0.0001 for p75^{NTR} effects, # p<0.0001 and Ψ p<0.0for NGF effects.

### p75^{NTR} Mediated Signal Transduction

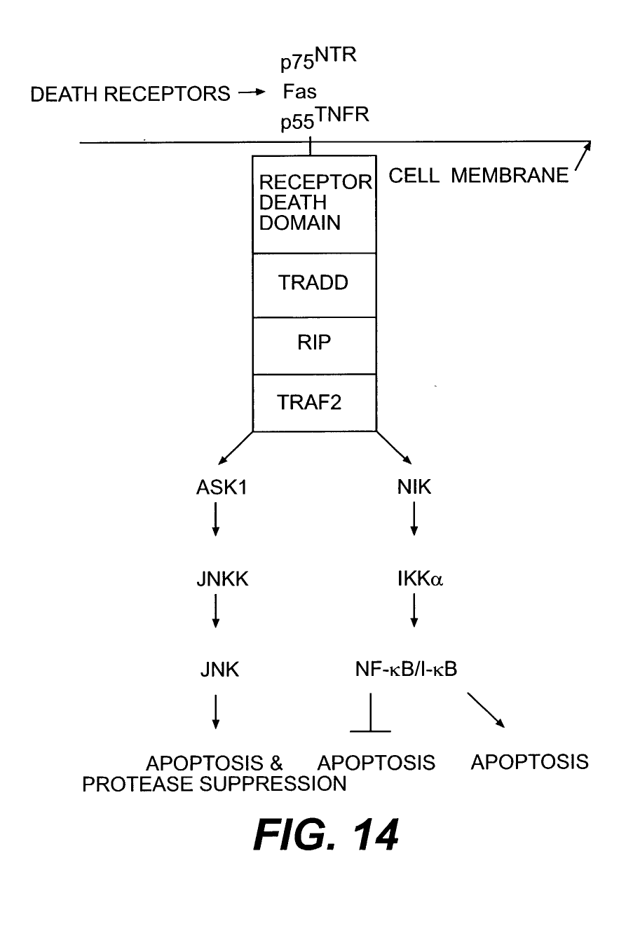

Elucidation of p75^{NTR} mediated signal transduction has been complicated by the observation that the p75^{NTR} lacks intrinsic kinase activity. In addition, disparate p75^{NTR} mediated signal transduction pathways have been shown to be both tissue specific and context specific. For instance, in neuronal cell systems the p75^{NTR} can induce sphingomyelin hydrolysis to ceramide resulting in apoptosis and inhibition of cell growth. Alternatively, p75^{NTR} has been shown to activate the MAP kinase (ERK1/2) pathway in PC12 cells, smooth muscle cells and pancreatic cancer cells. Moreover, an association between the p75^{NTR} mediated sphingomyelin-ceramide pathway and the MAP kinase pathway has been suggested to be a factor in determining cell fate. Our investigations failed to implicate either of these pathways in p75^{NTR} mediated inhibition of prostate cell growth, although, undoubtedly, they mediate p75^{NTR} independent signal transduction pathways in the prostate. A third signal transduction pathway involving death receptors (p55^{TNFR}, Fas, DR3, DR4, DR5), including the p75^{NTR} , also have been characterized in a variety of cells and tissues (FIG. 14).

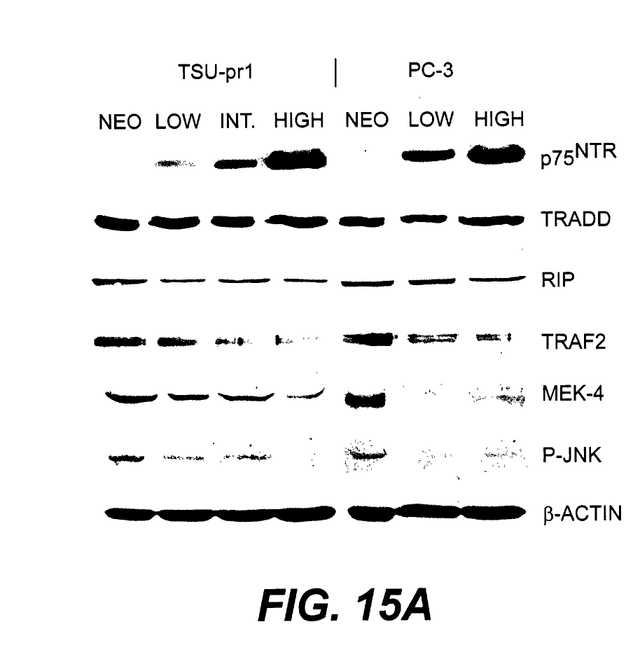

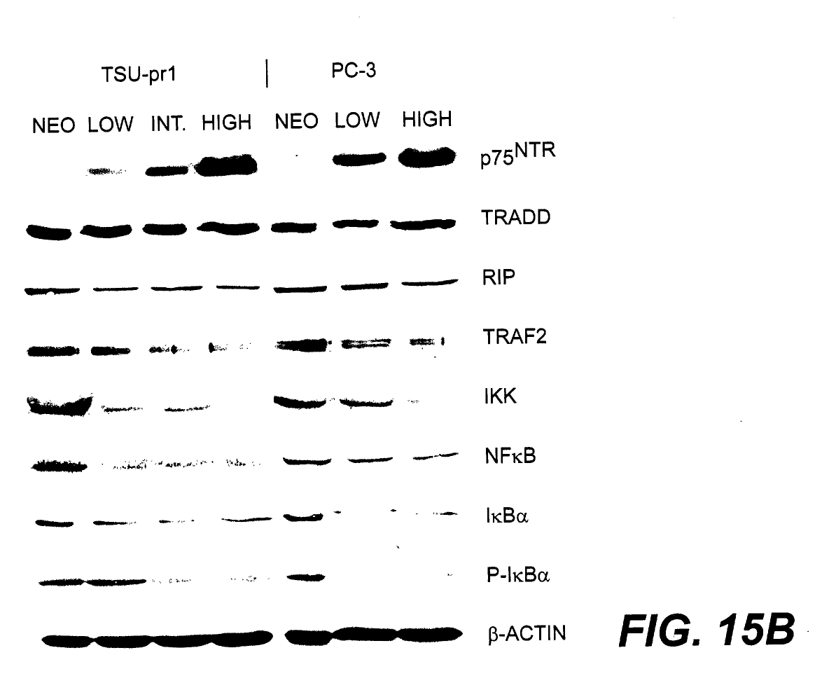

The death receptor signal transduction pathway (FIG. 14) is initiated following recruitment of the adapter protein TRADD (TNFR-associated death domain) to the death domain of the cytoplasmic receptor. TRADD subsequently binds the serine-threonine kinase RIP (receptor-interacting protein) that can then interact with TRAF2 (TNF receptor-associated factor-2). TRAF2 can activate NF-κB through stimulation of NF-κB inducing kinase (NIK) and I-κB kinase (IKKα). NF-κKB can dimerize with I-κB to induce apoptosis, whereas in the relative absence of I-κB, NF-κB can block apoptosis. Alternatively, TRAF2 has also been implicated in activation of c-Jun N-terminal kinase (JNK) via the apoptosis-inducing kinase (ASK1) and JNK kinase (JNKK). This JNK pathway may also transduce an apoptotic signal and a metastasis suppressor signal via modulation of cell migration and MMP-9 secretion (21). In order determine whether p75^{NTR} mediated signal transduction occurred via these bifurcating components of the death receptor pathway (FIG. 14), we have investigated some of the changes in the expression of components of the death receptor pathway (FIG. 15) associated with a rank order (dose-dependent) increase in expression of the p75^{NTR} protein in both PC-3 clones and TSU-pr1 clones. FIG. 15 shows that a rank order (dose-dependent) increase in the expression of p75^{NTR} protein in both the PC-3 and TSU-pr1 clones was associated with a concomitant decrease in the expression of RIP, TRAF2, IKK, NFκB and IκBα (right panel) associated with induction of apoptosis (tumor suppressor function) and also a concomitant decrease in the expression of RIP, TRAF2, MEK-4 and phospho-JNK (left panel) associated with induction of apoptosis (tumor suppressor function) and inhibition of MMP-9 expression (metastasis suppressor function).

These results demonstrate a signal transduction pathway (FIGS. 14 & 15) originating from the p75^{NTR} which can mediate both a tumor suppressor function and a metastasis suppressor function in tumor cells.

### p75^{NTR} Regulation of the Cell Cycle

The results discussed above demonstrated that p75^{NTR} dependent inhibition of prostate tumor cell growth is associated with changes in the cell cycle whereby p75^{NTR} expression induces an increased accumulation of cells in G0-G1 and reduction of cells the S phase consistent with increased cell cycle quiescence. This is consistent with p75^{NTR} dependent inhibition of proliferation of tumor cells. In order to investigate the molecular mechanism by which p75^{NTR} impedes cell cycle dependent proliferation we have examined specific molecules associated with compoentns of the cell cycle.

The cell cycle is regulated by a holoenzyme complex of cyclins that act as regulatory subunits, and cyclin dependent kinases (cdks) that act as catalytic subunits to phosphorylate and inactivate the retinoblastoma protein (pRb) that then facilitates progression through the G1/S restriction point of the cell cycle. The activity of the cyclin/cdk holoenzyme complex is further regulated by the proliferating cell nuclear antigen (PCNA) that binds cyclin D1 and promotes progression through G1 into the S phase of the cell cycle.

Additionally, two broad families of cdk-inhibitory proteins, the Ink4s and the Cip/KIPs, inhibit holoenzyme activity and cell cycle progression. In general the expression of cyclin D-cdk4/6 complexed with PCNA promotes phosphorylation of pRb during early to mid G1, expression of cyclin E-cdk2 promotes phosphorylation of pRb near the end of G1, and expression of cyclin A-cdk2 maintains phosphorylation of pRb during S phase (36). The accumulation of these cyclin/cdk complexes promote and maintain phosphorylation of pRb, which in a phosphorylated state is inactivated and can no longer function as a growth suppressor.

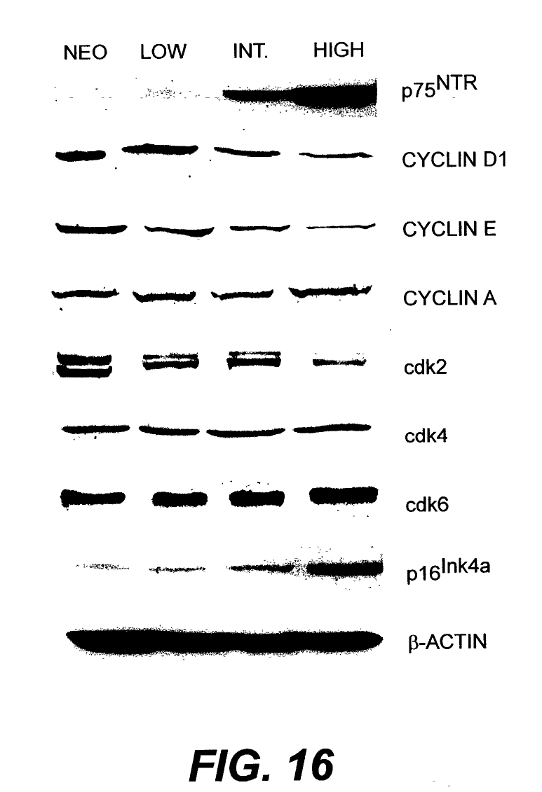

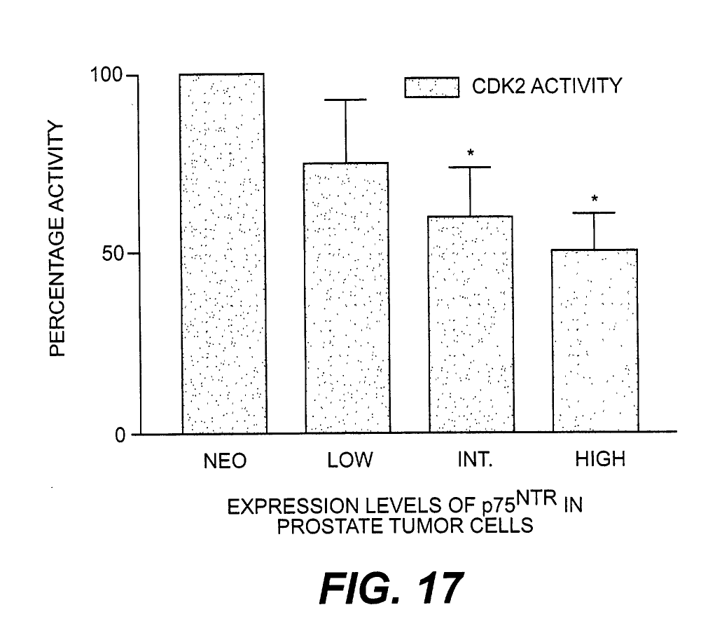

In order to provide preliminary results to support the rationale and feasibility of the second specific aim we investigated some changes in expression of components of the cyclin/cdk holoenzyme complexes associated with a rank order (dose-dependent) increase in expression of the p75^{NTR} protein in the TSU-pr1 clones. It is clear from FIG. 16 that a rank order increase in p75^{NTR} protein was associated with a concomitant reduction in the expression of cyclin D1, cyclin E, and cdk2, with no change in cyclin A, and a reduction in p16^{Ink4a} expression. In addition, the activity of CDK2 (FIG. 17) declined in response to increased expression of the p75^{NTR} protein.

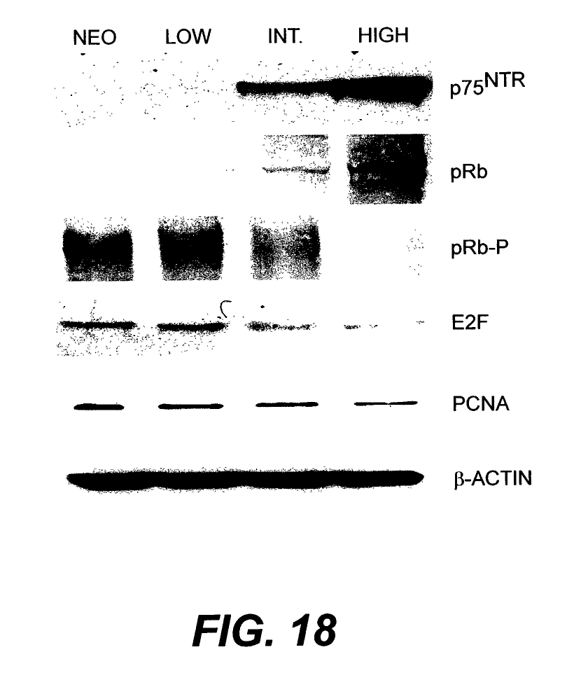

These results clearly demonstrate that increased p75^{NTR} protein expression is associated with changes in components of the cyclin/cdk holoenzyme complex consistent with cell cycle arrest. Moreover, a dose-dependent increase in expression of the p75^{NTR} protein was associated with increased expression of the Rb tumor suppressor protein (FIG. 18). During cell cycle arrest the cdk/cyclin complexes are prevented from phosphorylating the retinoblastoma protein (pRb). Unphosphorylated Rb is activated so that it can bind the E2F transcription factor. Bound E2F can no longer promote transcription of the proliferating cell nuclear antigen (PCNA) preventing progression into the S phase of the cell cycle. These results (FIG. 18) showing that increased p75^{NTR} protein expression is associated with increased Rb protein, reduced phosphorylation of the Rb protein, reduced E2F expression and reduced PCNA expression are all consistent with cell cycle arrest by preventing progression into the S phase of the cell cycle. These results are consistent with our working hypothesis that the p75^{NTR} gene product functions as a tumor suppressor in the human prostate by altering cell cycle kinetics, thereby providing direct support for the provisional patent application.

### p75^{NTR} Mediated Caspase Induction of Apoptosis

Apoptosis is a complex morphological and biochemical process that varies between tissues and cell type. Induction of mitochondrial stress via a number of mechanisms, including potentiation via death receptors, can induce the release of cytochrome c that initiates formation of the apoptosome and activation of a caspase cascade leading to apoptosis. The specific pathway of caspase activation is both tissue specific and context specific.

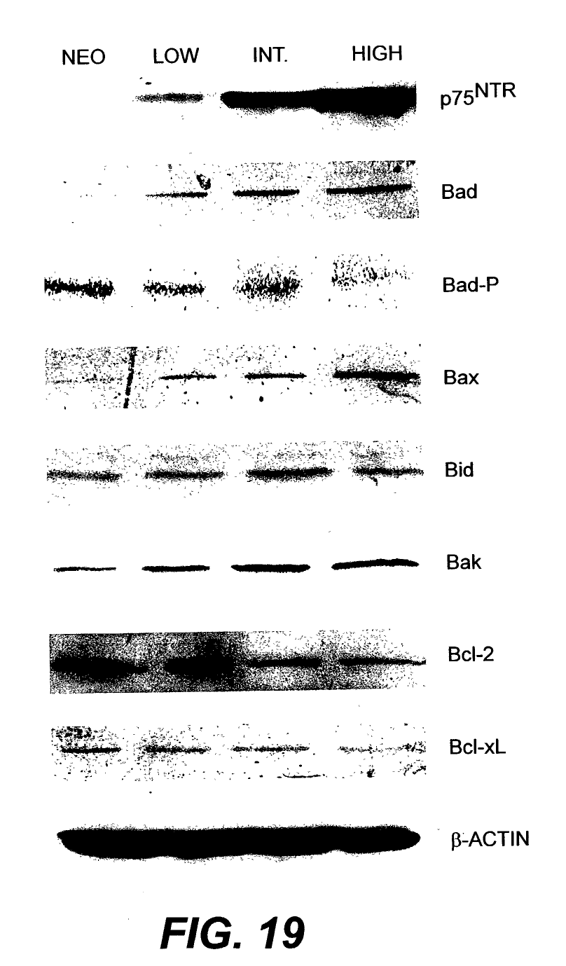

The action of pro-apoptotic effectors including the Bax, Bad, Bak and Bid molecules can be antagonized by a group of anti-apoptotic (pro-survival) molecules including Bcl-2 and Bcl-X_{L}. Hence, we investigated changes in expression of some apoptotic effectors associated with a rank order (dose-dependent) increase in expression of the p75^{NTR} protein in the TSU-pr1 clones. It is clear from FIG. 19 that a rank order increase in p75^{NTR} protein was associated with a concomitant increase in the expression of Bad, Bax, Bid and Bak, all of which are pro-apoptotic effectors, and reduced expression of phosphorylated Bad which can no longer dimerize, as well as Bcl-2 and Bcl-xL, which are anti-apoptotic effectors.

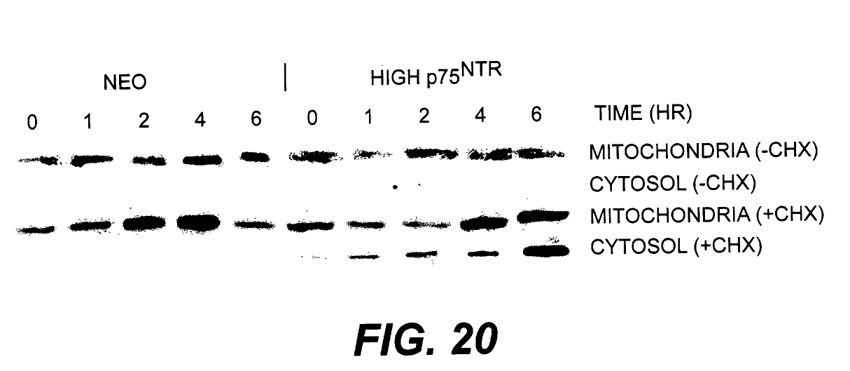

Alterations in mitochondrial membrane permeability following upregulation of pro-apoptotic signals (e.g. Bad and Bax) facilitates the release of cytochrome c, which in turn can complex with Apaf-1 and procaspase-9 in the apoptosome complex. It is clear from FIG. 20 that p75^{NTR} expression potyentiates release of cytochrome c from mitochondria.

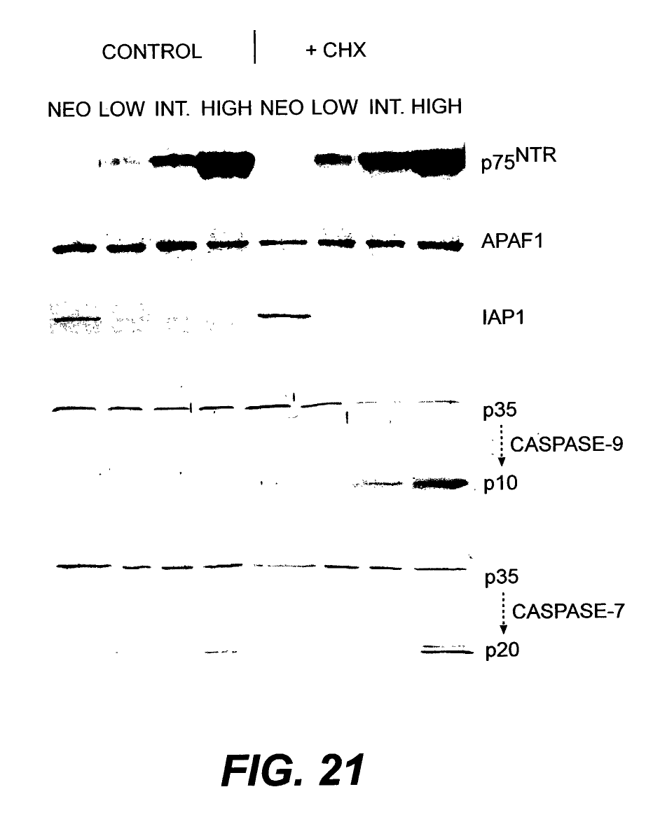

Activated caspase-9 is an initiator caspase that can activate downstream effector caspases by proteolytic processing. This apoptotic cascade can be antagonized by inhibitors of apoptosis proteins (IAPs). In the cytochrome c-dependent pathway (Apafs), IAPs exert their effects through direct interaction with procaspase-9, by competing for Apaf-1 binding to death domains, and through direct inhibition of active caspases. Since initiator caspases (e.g. caspase-9) are specific for each pathway, whereas effector caspases are often shared, we examined the effect of increased rank-order expression of p75^{NTR} protein on the activation of procaspase-9 to caspase-9 in tumor cells. Following standard procedures, apoptosis in these tumors cells was potentiated in the presence of cyclohexamide. It is clear a rank order increase in p75^{NTR} protein expression was associated with a concomitant reduction in IAP1 and activation of caspase-9 (FIG. 21). Activation was demonstrated by cleavage of the 35 kDa procaspase-9 molecule to generate the active 10 kDa subunit of caspase-9. FIG. 21 shows that a rank order increase in p75^{NTR} protein expression was also associated with a concomitant activation of a downstream effector caspase-7. The constitutive activation of procaspase-7 in the absence of cyclohexamide was observed in the high p75^{NTR} expression clones, whereas in the presence of cyclohexamide activation was potentiated by the further cleavage of the 35 kDa procaspase-7 molecule to generate the active 20 kDa subunit of caspase-7.

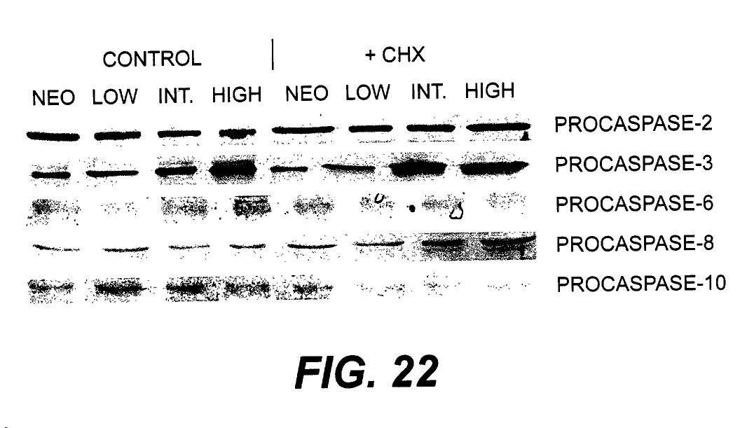

It is clear from FIG. 21 that a dose-dependent increase in p75^{NTR} protein was associated with a concomitant increase in the cyclohexamide potentiated activation of the apoptosome complex which initiated cleavage of procaspase-9 to yield its 10 kDa cleavage product, and the cleavage of procaspase-7 to yield its 20 kDa cleavage product. Other caspases did not appear to be activated by p75^{NTR} expression or cyclohexamide (FIG. 22).

These results are consistent with our working hypothesis that the p75^{NTR} gene product functions as a tumor suppressor in the human prostate, in part, by inducing specific caspase activated apoptosis, thereby providing direct support for the provisional patent application.

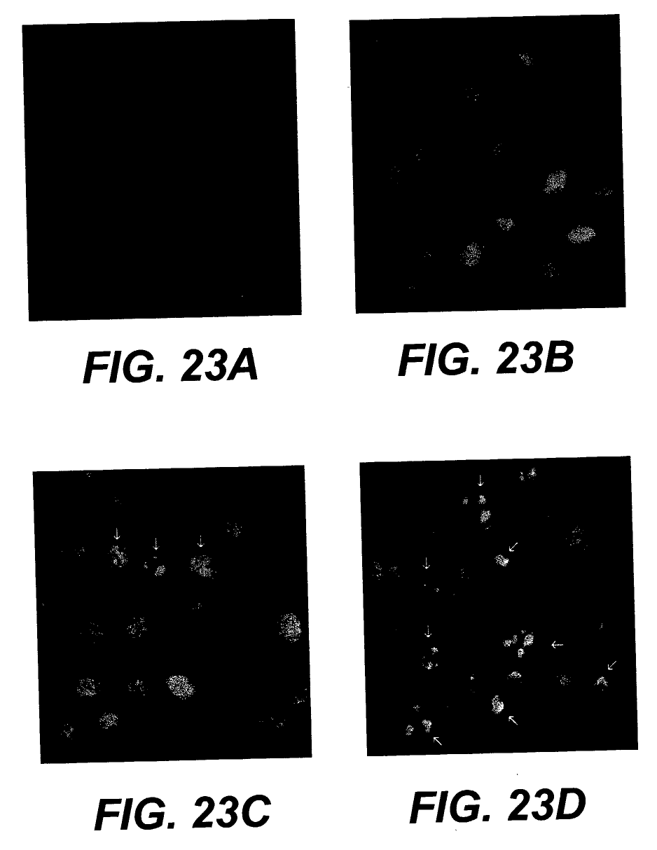

The final proof that p75^{NTR} can induce apoptosis is demonstrated by staining of nuclear fragmentation using Hoechst stain (FIG. 23). Tumor cells that do not express p75^{NTR} in the absence of cyclohexamide (FIG. 23A) or in the presence of cyclohexamide (FIG. 23B) did not exhibit nuclear fragmentation. However, cells that express high levels of p75^{NTR} exhibited nuclear fragmentation (arrows) consistent with induction of apoptosis (FIG. 23C). This process of p75^{NTR} mediated apoptosis was further potentiated in the presence of cyclohexamide (FIG. 23D). Hence, it is clear that p75^{NTR} can induce apoptosis in tumor cells. These results support the concept that the p75^{NTR} gene product functions as a tumor suppressor in the human prostate by inducing apoptosis, thereby providing direct support for the subject invention.

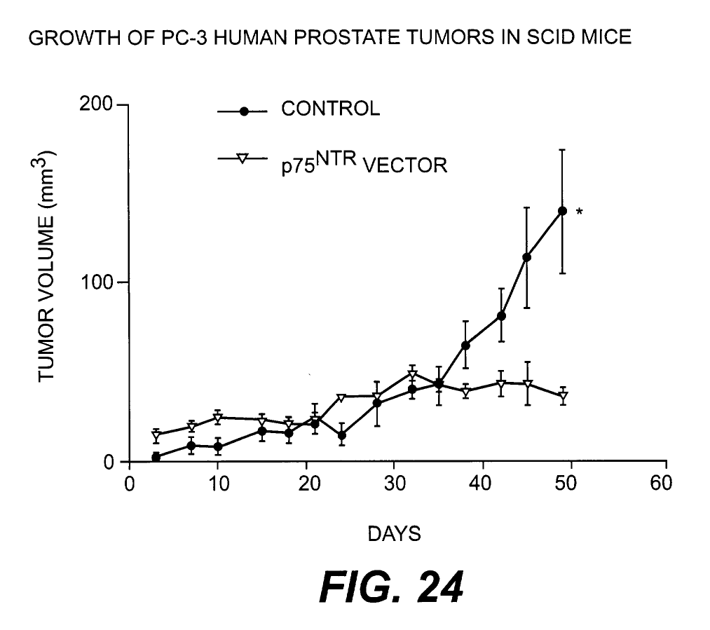

Since we have shown that the p75^{NTR} can inhibit growth by inhibiting cell cycle mediated growth and induction of apoptosis we carried out proof of concept gene therapy studies by growing PC-3 human prostate tumors in the flanks of severe combined immunodeficient (SCID) mice and injecting a liposome encapsulated p75^{NTR} expression vector into the tumors. In this context we utilized a p75^{NTR} cDNA expression vector under CMV promotion that we had previously show can induce expression of the protein in tumor cells (FIGS. 15,16,18,19,21). After allowing PC-3 human prostate tumors to grow in the flanks of SCID mice for 10 days and then injecting 10 μg of DNA vector encapsulated in a liposome twice per week into the tumor, or liposome without p75 cDNA vector (control) injected into the tumors, it is clear that the p75^{NTR} expression vector inhibited growth of the PC-3 prostate tumors compared with control tumors (p<0.01) that were injected with liposome alone (FIG. 24). These results provide ample illustration for the efficacy of the subkect invention which is based on the unexpected discovery that the p75^{NTR} is a tumor suppressor gene can be used with great efficacy in gene therapy of prostate cancer cells to prevent growth of prostate tumors.

The genetic materials according to the invention can be administered into target cells with or without the use of vectors or carriers. For example, genetic material can be introduced systemically through an intravenous or intraperitoneal injection for in vivo applications, or can be introduced to the site of action by direct injection into that area. However, DNA by itself is hydrophilic, and the hydrophobic character of the cellular membrane poses a significant barrier to the transfer of DNA across it. Accordingly, it has become preferred in the art to use facilitators that enhance the transfer of DNA into cells on direct injection.

The complexity of vectors that are capable of carrying DNA into cells ranges from plasmids, independent self-replicating circular DNA molecules, to adeno and herpes viruses. Typically, genetic engineering is used to modify the viral genes to make viruses incapable of replication.

Various vectors have been developed to deliver genes to cancer cells for expression of cytotoxic or radiation sensitizing agents. The delivery of these vectors has frequently employed direct injection of virus containing solutions into tumors. This intratumoral delivery of genes may involve injection into single or multiple locations throughout the tumor volume. The delivery of genes or cytokines into a tumor offers a particularly attractive option.

Other methods for effecting gene delivery include, by way of example liposomal delivery systems, the introduction of cells that express desired nucleic acid sequences, and the direct injection of naked DNA, e.g., viruses or antisense oligonucleotides at a target site, e.g., a tumor

Another approach in the art to delivery of genetic material to target cells is one that takes advantage of natural receptor-mediated endocytosis pathways that exist in such cells. Several cellular receptors have been identified heretofore as desirable agents by means of which it is possible to achieve the specific targeting of drugs, and especially macromolecules and molecular conjugates serving as carriers of genetic material of the type with which the present invention is concerned. These cellular receptors allow for specific targeting by virtue of being localized to a particular tissue or by having an enhanced avidity for, or activity in a particular tissue. This affords the advantages of lower doses or significantly fewer undesirable side effects. It has also been proposed in the art of receptor-mediated gene transfer that in order for the process to be efficient in vivo, the assembly of the DNA complex should result in condensation of the DNA to a size suitable for uptake via an endocytic pathway.

An alternative method of providing cell-selective binding is to attach an entity with an ability to bind to the cell type of interest; commonly used in this respect are antibodies which can bind to specific proteins present in the cellular membranes or outer regions of the target cells. Alternative receptors have also been recognized as useful in facilitating the transport of macromolecules, such as biotin and folate receptors; transferrin receptors; insulin receptors; and mannose receptors. The enumerated receptors are merely representative, and other examples will readily come to the mind of the artisan.

The conjugation of different functionalities on the same molecule has also been utilized in the art. The method consists of attaching a glycoprotein, asialoorosomucoid, to poly-lysine to provide a hepatocyte selective DNA carrier. The function of the poly-lysine is to bind to the DNA through ionic interactions between the positively charged (cationic) amino groups of the iysines and the negatively charged (anionic) phosphate groups of the DNA. Orosomucoid is a glycoprotein which is normally present in human serum. Removal of the terminal sialic acid (N-acetyl neuraminic acid) from the branched oligosaccharides exposes terminal galactose oligosaccharides, for which hepatocyte receptors have a high affinity, as already described.

After binding to the asialoglycoprotein receptor on hepatocytes, the protein is taken into the cell by endocytosis into a pre-lysosomal endosome. The DNA, ionically bound to the poly-lysine-asialoorosomucoid carrier, is also taken into the endosome. Partial hepatectomy improves the rsistence of the expression of the DNA delivered into the hepatocytes. The transfer of the DNA into cells by this mechanism is also significantly enhanced by the addition of cationic lipids. The use of a specific asialoglycoprotein is not required to achieve binding to the asialoglycoprotein receptor; this binding can also be accomplished with high affinity by the use of small, synthetic molecules having a similar configuration. The carbohydrate portion can be removed from an appropriate glycoprotein and be conjugated to other macromolecules. By this procedure the cellular receptor binding portion of the glycoprotein is removed, and the specific portion required for selective cellular binding can be transferred to another molecule. Reductive amination of a peptide with a branched tri-lysine amino terminus gives a ligand ending with four galactosyl residues that can be readily coupled to poly-lysine or other macromolecules and has been used to prepare DNA constructs.

Thiopropionate and thiohexanoate glycosidic derivatives of galactose have been prepared and linked to L-lysyl-L-lysine to form a synthetic tri-antennary galactose derivative. A bisacridine spermidine derivative containing this synthetic tri-antennary galactose has been used to target DNA to hepatocytes.

Other means of providing cellular receptor based facilitation of gene transfer into cells using poly-lysine as a carrier have been described in the art. Antibodies specific for cell surface thrombomodulin have been used with poly-lysine as a delivery system for DNA in vitro and in vivo. The transferrin receptor has also been used to target DNA to erythroblasts, K562 macrophages and ML-60 leukemic cells, both small oliogodeoxynucleotides as well as large plasmids are used.

The ability of poly-lysine to facilitate DNA entry into cells is significantly enhanced if the poly-lysine is chemically modified with hydrophobic appendages; see X. Zhou and L. Huang, Biochim. Biophys. Acta, 1189, 195-203 (1994); complexed with cationic lipids; see K. D. Mack, R. Walzem and J. B. Zeldis, Am. J. Med. Sci., 307,138-143 (1994) or associated with viruses. Many viruses infect specific cells by receptor mediated binding and insertion of the viral DNA/RNA into the cell; and thus this action of the virus is similar to the facilitated entry of DNA described above.

Replication-incompetent adenovirus has been used to enhance the entry of transferrin-poly-lysine complexed DNA into cells. The adenovirus enhances the entry of the poly-lysine-transferrin-DNA complex when covalently attached to the poly-lysine and when attached through an antibody binding site. There does not need to be a direct attachment of the adenovirus to the poly-lysine-transferrin-DNA complex, and it can facilitate the entry of the complex when present as a simple mixture. The poly-lysine transferrin-DNA complex provides receptor specific binding to the cells and is internalized into endosomes along with the DNA. Once inside the endosomes, the adenovirus facilitates entry of the DNA/transferrin-poly-lysine complex into the cell by disruption of the endosomal compartment with subsequent release of the DNA into the cytoplasm. Replication-incompetent adenovirus has also been used to enhance the entry of uncomplexed DNA plasmids into cells without the benefit of the cell receptor selectivity conferred by the poly-lysine-transferrin complex.

Synthetic peptides such as the N-terminus region of the influenza hemagglutinin protein are known to destabilize membranes and are known as fusogenic peptides. Conjugates containing the influenza fusogenic peptide coupled to poly-lysine together with a peptide having a branched tri-lysine amino terminus ligand ending with four galactosyl residues have been prepared as facilitators of DNA entry into hepatocytes. These conjugates combine the asialoglycoprotein receptor mediated binding conferred by the tetra-galactose peptide, the endosomal disrupting abilities of the influenza fusogenic peptide, and the DNA binding of the poly-lysine. These conjugates deliver DNA into the cell by a combination of receptor mediated uptake and internalization into endosomes. This internalization is followed by disruption of the endosomes by the influenza fusogenic peptide to release the DNA into the cytoplasm. In a similar fashion, the influenza fusogenic peptide can be attached to poly-lysine and mixed with the transferrin-poly-lysine complex to provide a similar DNA carrier selective for cells carrying the transferrin receptor. Synthetically designed peptides can also be used. The cationic amphipathic peptide gramicidin S can facilitate entry of DNA into cells, but also requires a phospholipid to achieve significant transfer of DNA.

Poly-lysine is not unique in providing a polycationic framework for the entry of DNA into cells. DEAE-dextran has also been shown to be effective in promoting RNA and DNA entry into cells; More recently, a dendritic cascade co-polymer of ethylenediamine and methyl acrylate has been shown to be useful in providing a carrier of DNA which facilitates entry into cells; see J. Haensler and F. C. Szoka, Jr., Bioconj. Chem., 4, 372-379 (1993). An alkylated polyvinylpyridine polymer has also been used to facilitate DNA entry into cells; see A. V. Kabanov, I. V. Astafieva, I. V. Maksimova, E. M. Lukanidin, G. P. Georgiev and V. A. Kabanov, Bioconj. Chem., 4, 448-454 (1993). Positively charged liposomes have also been widely used as carriers of DNA which facilitate entry into cells. These carrier compositions have also included pH sensitive liposomes. A poly-cationic lipid has been prepared by coupling dioctadecylamidoglycine and dipalmitoyl phosphatidylethanolamine to a 5-carboxyspermine. These lipophilic-spermines are very active in transferring DNA through cellular membranes.

Combinations of lipids have been used to facilitate the transfer of nucleic acids into cells. For example, in U.S. Pat. No. 5,283,185 there is disclosed such a method which utilizes a mixed lipid dispersion of a cationic lipid with a co-lipid in a suitable solvent. The lipid has a structure which includes a lipophilic group derived from chlolesterol, a linker bond, a linear alkyl spacer arm, and a cationic amino group; and the co-lipid is phosphatidylcholine or phosphatidylethanolamine.

The present invention contemplates the use of p75^{NTR} in gene therapy in combination with prostate tumor cell apoptosis promoters in order to suppress the growth of prostate tumors. The delivery Pharmaceutical Compositions and Routes of Administration. Compositions of the present invention will have an effective amount of a gene for therapeutic administration, optionally in combination with an effective amount of a compound (second agent) that is a chemotherapeutic agent. Such compositions will generally be dissolved or dispersed in a pharmaceutically acceptable carrier or aqueous medium.

The expression vectors and delivery vehicles of the present invention may include classic pharmaceutical preparations. Administration of these compositions according to the present invention will be via any common route so long as the target tissue is available via that route. This includes oral, nasal, buccal, rectal, or topical. Alternatively, administration may be by orthotopic, intradermal, subcutaneous, intramuscular, intraperitoneal or intravenous injection. Such compositions would normally be administered as pharmaceutically acceptable compositions, described supra.

The vectors of the present invention are advantageously administered in the form of injectable compositions either as liquid solutions or suspensions; solid forms suitable for solution in, or suspension in, liquid prior to injection also may be prepared. These preparations also may be emulsified. A typical compositions for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well known parameters.

Targeting of cancerous tissues underexpressing p75^{NTR} may be accomplished in any one of a variety of ways. Plasmid vectors and retroviral vectors, adenovirus vectors, and other viral vectors all present means by which to target human cancers. The inventors anticipate particular success for the use of liposomes to target p75^{NTR} genes to cancer cells. Of course, the potential for liposomes that are selectively taken up by a population of cancerous cells exists, and such liposomes will also be useful for targeting the gene.

Those of skill in the art will recognize that the best treatment regimens for using p75^{NTR} to suppress prostate cancers can be straightforwardly determined. This is not a question of experimentation, but rather one of optimization, which is routinely conducted in the medical arts. The in vivo studies in nude mice provide a starting point from which to begin to optimize the dosage and delivery regimes. The frequency of injection will initially be once a week. However, this frequency might be optimally adjusted from one day to every two weeks to monthly, depending upon the results obtained from- the initial clinical trials and the needs of a particular patient. Human dosage amounts can initially be determined by extrapolating from the amount of p75^{NTR} used in mice, approximately 15 μg of p75^{NTR} DNA per 50 g body weight. Based on this, a 100 kg man would require treatment with 30 mg of DNA per dose. In certain embodiments it is envisioned that this dosage may vary from between about 100 μg/50 g body weight to about 5 μg/g body weight; or from about 90 μg/50 g body weight to about 10 μg/g body weight or from about 80 μg/50 g body weight to about 15 μg/g body weight; or from about 75 μg/50 g body weight to about 20 μg/g body weight; or from about 60 μg/50 g body weight to about 30 μg/g body weight about 50 μg/50 g body weight to about 40 μg/g body weight. In other embodiments this dose may be about 5, 8, 10 15, or 20 μg/50 g. Of course, this dosage amount may be adjusted upward or downward, as is routinely done in such treatment protocols, depending on the results of the initial clinical trials and the needs of a particular patient.

While the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the following claims, including equivalents thereof. In the above description and the claims below, p75^{NTR} gene is intended to represent not only the p75^{NTR} gene but also all the homologs, allelic variants, synthetic variants with 80%, 90%, 95%, and 97% sequence identity. A fragment of the p75^{NTR} gene is any fragment capable of promoting p75^{NTR} expression.

### REFERENCES

1. Landis, S. M. (1998) CA. Cancer J. Clin. 48, 6-29

2. Arason, A., Barkardottir, R. B., & Egilisson, V. (1993) Am. J. Hum. Gen. 17, 618-623.

3. Ford, D., Easton, D. F., Bishop, D. T., Narod S. A. & Goldgar, D. E. (1994) Lancet 343 (8899), 692-695.

4. Gao, X., Zacharek, A., Grignon, D. J., Sakr, W., Powell, I. J., Porter, A. T. & Honn, K. V. (1995) Oncogene 11, 1241-1247.

5. Murakami, Y. S., Brothman, A. R., Leach, R. J. & White, R. L. (1995) Canc. Res. 55, 3389-3394.

6. Gao, X., Zacharek, A., Salkowski, A., Grignon, D. J., Sakr, W., Porter, A. T. & Honn, K. V. (1995) Canc. Res. 55, 1002-1005.

7. Williams, B. J., Jones, E., Zhu, X. L., Steele, M. R., Stephensen, R. A., Rohr, L. R. & Brothman, A. R. (1996) J. Urol. 155, 720-725.

8. Huebner, K., Isobr, M., Chao, M., Bothwell, M., Ross, A. L., Finan, J., Hoxie, J. A., Sethgal, A., Buck, C. R., Lanahan, A., Nowell, P. C., Koprowski H. & Croce, C. M. (1986) Proc. Nati. Acad. Sci. USA 83,1403-1407.

9. Pflug, B. R., Onoda, M., Lynch J. H., & Djakiew, D. (1992) Canc. Res. 52, 5403-5406,

10. Graham, C., Lynch, J. H. & Djakiew, D. (1992) J. Urol. 147,1444-1447.

12. Pflug, B., Dionne, C., Kaplan, D., Lynch J., & Djakiew, D. (1995) Endocrinology 136, 262-268.

13. Perez, M., Regan, T., Pflug, B., Lynch J. & Djakiew, D. (1997) Prostate 30, 274-279.

14. Barrett, G. L. & Bartlett, P. F. (1994) Proc. Natl. Acad. Sci USA 91, 6501-6505.

15. Rabizadeh, S., Oh, J., Zhong, L. T., Yang, J., Bitler, C. M., Butcher, L. L. & Bresden, D. E. (1993) Science 261, 345-348.

16. Pflug, B. & Djakiew, D. (1998) Mol. Carcinogenesis 23, 106-114.

17. Vindelov, L. L., Christensen, I. S. & Nissen, N. I. (1983) Cytochemistry 3, 323-327.

18. Tomayko, M. M. & Reynolds, C. P. (1989) Cancer Chemother. Pharmacol. 24, 148-154.

19. Gee, J. M. W., Robertson, J. F. R., Ellis, I. O., Nicholson, R. I. & Hurst, H. C. (1999) J. Path. 189, 514-520.

20. Al-Tubuly, A. A., Spijker, R., Pignatelli, M., Kirkland, S. C. & Pitter, M. A. (1997) Int. J. Cancer 71, 605-611.

21. Moretti, F., Fassetti, A., Soddu, S., Misiti, S., Crescenzi, M., Filetti, S., Andreoli, M., Sacchi, A. & Pontecorvi, A. (1997) Oncogene 14, 729-740.

22. Levine, A. J. (1997) Cell 88, 323-331.

23. Zhu, J., Jiang, J., Zhou, W. & Chen, X. (1998) Canc. Res. 58, 5061-5065.

24. Le Dai, J. Bansal, R. K. & Kern, S. E. (1999)) Proc. Natl. Acad. Sci USA 96, 1427-1432.

25. Hall, M. C., Li, Y., Pong, R. C., Ely, B., Sagalowsky, A. I. & Hsieh, J. T. (2000) J. Urol. 163, 1033-1038.

26. Chao, M. V. (1994). J. Neurobiol. 25, 1373-1385.

27. Chapman, B. S. (1995). F.E.B.S. Letters 374, 216-220.

28. Pai, S. I., Wu, G. S., Ozoren, N., Wu, L., Jen, J., Sidransky, D. & El-Deiry, W. S. (1998) Canc. Res. 58, 3513-3518.

29. Lee, K. F., Bachman, K., Landis, S. & Jaenisch, R. (1994) Science 263, 1447-1449.

30. Frade, J. M., Rodriguez-Tebar, A. & Barde, Y. A. (1996) Nature 383, 166-168.

31. Bunone, G., Mariotti, A., Compagni, A., Morandi, E. & Valle, G. D. (1997) Oncogene 14, 1463-1470.

32. Al, L. S., CHAU, L. Y., Post-transcriptional regulation of H-ferritin mRNA. J. Biol. Chem., 274, 30209-30214 (1999).

33. AMARA, F. M., CHEN, F. Y., and WRIGHT, J. A., Defining a novel cis element in the 3′-untranslated region of mammalian ribonucleotide reductase component R2 mRNA: role in transforming growth factor-β₁ induced mRNA stabilization. Nucleic Acids Res., 23, 1461-1467 (1995).

34. AMARA, F. M., SUN, J., and WRIGHT, J. A., Defining a novel cis-element in the 3′-untranslated region of mammalian ribonucleotide reductase component R2 mRNA. J. Biol. Chem., 271, 20126-20131 (1996a).

35. AMARA, F. M., ENTWISTLE, J., KUSCHAK, T. I, TURLEY, E. A., and WRIGHT, J. A., Transforming growth factor-β₁ stimulates multiple protein interactions at a unique cis-element in the 3′-untranslated region of the hyaluronan receptor RHAMM mRNA. J. Biol. Chem., 271, 15279-15284 (1996b).

36. BUNONE, G., MARIOTTI, A., COMPAGNI, A., MORANDI, E., and VALLE, G. D., Induction of apoptosis by p75^{NTR} neurotrophin receptor in human neuroblastoma cells. Oncogene, 14,1463-1470 (1997).

37. CAMPOS-CARO, A., CARRASCO-SERRANO, C., VALOR, L. M., VINIEGRA, S., BALLESTA, J. J., and CRIADO, M., Mutiple functional Sp1 domains in the minimal promoter region of the neuronal nicotinic receptor α5 subunit gene. J. Biol. Chem., 274, 4693-4701 (1999).

38. CARRASCO-SERRANO, C., CAMPOS-CARO, A., VINIEGRA, S., BALLESTA, J. J., and CRIADO, M., GC- and E-box motifs as regulatory elements in the proximal promoter region of the neuronal nicotinic receptor α7 subunit gene. J. Biol. Chem., 27, 20021-20028 (1998).

39. CHAO, M. V., BOTHWELL, M. A., ROSS, A. H., KOPROWSKI, H., LANAHAN, A. A., BUCK, C. R., and SEHGAL, A., Gene transfer and molecular cloning of the human NGF receptor. Science, 230, 518-521 (1986).

40. CHIARAMELLO, A., NEUMAN, K., PALM, K., METSIS, M., and NEUMAN, T., Helix-loop-helix transcription factors mediate activation and repression of the p75LNGFR gene. Mol. Cell Biol., 15, 6036-6044 (1995).

41. COLLINS, M. and BORNSTEIN, P., Sp1-binding elements, within the common metaxin-thrombospondin 3 intergenic region, participate in the regulation of the metaxin gene. Nucleic Acids Res., 24, 3661-3669 (1996).

42. DJAKIEW, D., DELSITE, R., DALAL, R., and PFLUG, B., The role of the low affinity nerve growth factor receptor and the high affinity Trk receptor in human prostate carcinogenesis. Radiat. Oncol. Invest., 3, 333-339 (1996).

43. DJAKIEW, D., Dysregulated expression of growth factors and their receptors in the development of prostate cancer. Prostate 42,150-160 (2000).

44. DIONNE, C., CAMORATTO, A. M., JANI, J., EMERSON, E., NEFF, N. T., VAUGHT, J., MURAKATA, C., DJAKIEW, D., LAMB, J., BOVA, S., GEORGE, D., and ISSACS, J., Cell-cycle independent death of prostate adenocarcinoma is induced by the Trk tyrosine kinase inhibitor CEP-751 (KT6587). Clin. Cancer Res., 4,1887-1898 (1998).

45. DYNAN, W. S. and TIJAN, R., The promoter-specific transcription factor Sp1 binds to upstream sequences in the SV40 early promoter. Cell, 35, 79-87 (1983).

46. FABRICANT, R. N., DELARCO, J. E., and TODARO, G. J., Nerve growth factor receptors on human melanoma cells in culture. Proc. Natl. Acad. Sci. USA, 74, 565-569 (1977).

47. FRADE, J. M., RODRÍGUEZ-TÉBAR, A., and BARDE, Y. A., Induction of cell death by endogenous nerve growth factor through its p75 receptor. Nature, 383, 166-168 (1996).

48. GAO, X., ZACHAREK, A., SALKOWSKI, A., GRIGNON, D. J., SAKR, W., PORTER, A. T., and HONN, K. V., Loss of heterozygosity of the BRCA1 and other loci on chromosome 17q in human prostate cancer. Cancer Res., 55, 1002-1005 (1995a).

49. GAO, X., ZACHAREK, A., GRIGNON, D. J., SAKR, W., POWELL, I. J., PORTER, A. T., and HONN, K. V., Localization of potential tumor suppressor loci to a <2 Mb region on chromosome 17q in human prostate cancer. Oncogene, 11, 1241-1247 (1995b).

50. HEW, Y., GRZELCZAK, Z, LAU, C., and KEELEY, F. W., Identification of a large region of secondary structure in the 3′-untranslated region of chicken elastin mRNA with implications for the regulation of mRNA stability. J. Biol. Chem., 274, 14415-14221 (1999).

51. HUEBNER, K., ISOBE, M., CHAO, M., BOTHWELL, M., ROSS, A. H., FINAN, J., HOXIE, J. A., SEHGAL, A., BUCK, C. R., LANAHAN, A., NOWELL, P. C., KOPROWSKI, H., and CROCE, C. M., The nerve growth factor receptor gene is at human chromosome region 17q12-17q22, distal to the chromosome 17 breakpoint in acute leukemias. Proc. Natl. Acad. Sci. USA, 83, 1403-1407 (1986).

52. JOHNSON, D., LANAHAN, A., BUCK, C. R., SEHGAL, A., MORGAN, C., MERECER, E., BOTHWELL, M., and CHAO, M., Expression and structure of the human NGF receptor. Cell, 47, 545-554 (1986).

53. LALLE, P., DELATOUR, M., RIO, P., and BIGNON, Y. J., Detection of allelic losses on 17q12-q21 chromosomal region in benign lesions and malignant tumors occurring in a familial context. Oncogene, 9, 437-442 (1994).

54. LANDIS, S. H., Cancer Statistics, 1998, CA Cancer J. Clin., 48, 6-29 (1998).

55. LEE, K. F., BACHMAN, K., LANDIS, S., and JAENISCH, R., Dependence on p75 for innervation of some sympathetic targets. Science, 263, 1447-1449 (1994).

56. MADIREDDI, M. T., DENT, P., and FISHER, P. B., Regulation of mda-7 gene expression during human melanoma differentiation. Oncogene, 19, 1362-1368 (2000).

57. MCGOWAN, K. M., POLICE, S., WINSLOW, J. B., and PEKALA, P. H., Tumor necrosis factor-α regulation of glucose transporter (GLUT1) mRNA turnover. J. Biol. Chem., 272, 1331-1337 (1997).

58. MELTON, D. W., KONECKI, D. S., BRENNAD, J., and CASKEY, C. T., Structure, expression, and mutation of the hypoxanthine phosphoribosyltransferase gene. Proc. Natl. Acad. Sci. USA, 81, 2147-2151 (1984).

59. MÜLLNER, E. W. and KÜHN, L. C., A stem-loop in the 3′ untranslated region mediates iron-dependent regulation of transferrin receptor mRNA stability in the cytoplasm. Cell, 53, 815-825 (1988).

60. PEREZ, M., REGAN, T., PFLUG, B., LYNCH, J., and DJAKIEW, D., Loss of the low affinity nerve growth factor receptor during malignant transformation of the human prostate. Prostate, 30, 274-279 (1997).

61. PFLUG, B. R., ONODA, M., LYNCH, J. H., and DJAKIEW, D., Reduced expression of the low affinity nerve growth factor receptor in benign and malignant human prostate tissue and loss of expression in four human metastatic prostate tumor cell lines. Cancer Res., 52, 5403-5406 (1992).

62. PFLUG, B. R., DIONNE, C. A., KAPLAN, D. R., LYNCH, J. H., and DJAKIEW, D., Expression of the Trk high affinity nerve growth factor receptor in the human prostate. Endocrinology, 136, 262-268 (1995).

63. PFLUG, B. and DJAKIEW D., Expression of p75^{NTR} in a human prostate epithelial tumor cell line reduces nerve growth factor-induced cell growth by activation of programmed cell death. Mol. Carcinog., 23, 106-114 (1998).

64. POUKKA H., KALLIO, P. J., JÄNNE, and PALVIMO, J. J., Regulation of the rat p75 neurotrophin receptor promoter by GC element binding proteins. Biochem. Biophys. Res. Comm., 229, 565-570 (1996).

65. RADEKE, M. J., MISKO, T. P., HSU, C., HERZENBERG, L. A., and SHOOTER, E. M., Gene transfer and molecular cloning of the rat nerve growth factor receptor. Nature, 325, 593-597 (1987).

66. RETTIG, W. J., THOMSON, T. M., SPENGLER, B. A., BIEDLER, J. L., and OLD, L. J., Assignment of human nerve growth factor receptor gene to chromosome 17 and regulation of receptor expression in somatic cell hybrids. Som. Cell Mol. Gen., 12, 441-447 (1986).

67. REYNOLDS, G. A., BASU, S. K., OSBORNE, T. F., CHIN, D. J., GIL, G., BROWN, M. S., GOLDSTEIN, J. L., and LUSKEY, K. L., HMG CoA reductase: a negatively regulated gene with unusual promoter and 5′ untranslated regions. Cell, 38, 275-285 (1984).

68. ROSS, A. H., GROB, P., BOTHWELL, M., ELDER, D. E., ERNST, C. S., MARANO, N., GHRIST, F. D., SLEMP, C., HERLYN, M., ATKINSON, B., and KOPROWSKI, H., Characterization of nerve growth factor receptor in neural crest tumors using monoclonal antibodies. Proc. Natl. Acad. Sci. USA, 81, 6681-6685 (1984).

69. SCHENONE, A., GILL, J. S., ZACHARIAS, D. A., and WINDEBANK, A. J., Expression of high- and low-affinity neurotrophin receptors on human transformed B lymphocytes. J. Neuroimmunology, 64,141-149 (1996).

70. SEHGAL, A., PATIL, N., and CHAO, M., A constituitive promoter directs expression of the nerve growth factor receptor gene. Mol. Cell Biol., 8, 3160-3167 (1988).

71. VALERIO, D., DUYVESTEYN, M. G., DEKKER, B. M., WEEDA, G., BERKVENS, T. M., VAN DER VOORN, L., VAN ORMONDT, H., and VAN DER EB, A. J., Adenosine deaminase: characterization and expression of a gene with a remarkable promoter. EMBO J., 4, 437-443 (1985).

72. VANTUINEN, P., RICH, D. C., SUMMERS, K. M., and LEDBETTER, D. H., Regional mapping panel for human chromosome 17: application to neurofibromatosis type I. Genomics, 1, 374-381 (1987).

73. WANG, F., WANG, W., and SAFE, S., Regulation of constituitive gene expression through interactions of Sp1 protein with the nuclear aryl hydrocarbon receptor complex. Biochemistry, 38, 11490-11500 (1999).

What is claimed is:

1. A method of treatment or prophylaxis of cancer in a subject in need thereof comprising administering to the subject p75^{NTR} gene or a fragment thereof in an amount effective to increase tumor suppression and/or tumor apoptosis.

2. The method of claim 1, wherein the p75^{NTR} gene or fragment thereof is administered in an amount sufficient to maintain a level of p75^{NTR} mRNA which at least partially compensates for the loss of p75^{NTR} mRNA associated with p75^{NTR} mRNA degradation in cancerous or precancerous cells.

3. The method of claim 1, wherein the p75^{NTR} gene or fragment thereof is administered in cDNA form.

4. The method of claim 1, wherein the p75^{NTR} gene or fragment thereof is administered directly in the form of naked DNA, in a liposomal delivery system or by a combination of receptor mediated uptake and internalization into endosomes.

5. The method of claim 1, wherein p75^{NTR} gene or fragment thereof is administered in an amount of about 100 μg/50 g body weight to about 5 μg/g body weight.

6. The method of claim 1, wherein p75^{NTR} gene or fragment thereof is administered in conjunction with a tumor cell apoptosis promoting agent.

7. The method of claim 1, wherein the p75^{NTR} gene or a fragment thereof is administered in an amount sufficient to induce G0/G1 cell cycle arrest.

8. The method of claim 1, wherein tumor suppression comprises decreased cell proliferation.

9. The method of claim 1, wherein increasing tumor apoptosis comprises reestablishing the apoptotic pathway associated with normal-cell p75^{NTR} gene expression.

10. The method of claim 1, wherein the cancer is prostate cancer.

11. The method of claim 10, wherein the p75^{NTR} gene or fragment thereof is administered in an amount sufficient to induce an accumulation of at least 56% of the tumor cells in the G0/G1 phase.

12. The method of claim 10, wherein the p75^{NTR} gene or fragment thereof is administered in an amount sufficient to induce an accumulation of at least 59% of the tumor cells in the G0/G1 phase.

13. The method of claim 10, wherein the p75^{NTR} gene or fragment thereof is administered in an amount sufficient to induce an accumulation of at least 68% of the tumor cells in the G0/G1 phase.

14. The method of claim 10, wherein the p75^{NTR} gene or fragment thereof is administered in an amount sufficient to induce an accumulation of at most 16% of the tumor cells in the G2-M phase and at most 28% in the S phase.

15. The method of claim 10, wherein the p75^{NTR} gene or fragment thereof is administered in an amount sufficient to induce an accumulation of at most 12% of the tumor cells in the G2-M phase and at most 28% in the S phase.

16. The method of claim 10, wherein the p75^{NTR} gene or fragment thereof is administered in an amount sufficient to induce an accumulation of at most 11% of the tumor cells in the G2-M phase and at most 21% in the S phase.

17. The method of claim 10, wherein the p75^{NTR} gene or fragment thereof is administered in an amount sufficient to reduce the percentage of proliferating tumor cells to about 42% or less.

18. The method of claim 10, wherein the p75^{NTR} gene or fragment thereof is administered in an amount sufficient to reduce the percentage of proliferating tumor cells to about 26% or less.

19. The method of claim 1, further comprising administering to the subject a p75^{NTR} mRNA stabilizing agent.

20. The method of claim 19, wherein the agent comprises one or more RNA-binding proteins.

21. The method of claim 19, wherein the agent is capable of regulating cell nutrients and/or cytokines associated with p75^{NTR} mRNA stability.

22. A method of treatment or prophylaxis of cancer in a subject in need thereof comprising administering to the subject a p75^{NTR}mRNA stabilizing agent.

23. The method of claim 22, wherein the agent comprises one or more RNA-binding proteins.

24. The method of claim 22, wherein the agent is capable of regulating cell levels of nutrients and/or cytokines associated with p75^{NTR} mRNA stability.

25. A method for early diagnosis of prostate cancer comprising determining p75^{NTR} mRNA levels in prostate tissue of a subject.

26. The method of claim 25, wherein determining p75^{NTR} mRNA levels in prostate tissue comprises isolating the RNA from the tissue; subjecting the RNA to reverse transcription and then to PCR amplification with a suitable primer; precipitating the product of the amplification reaction; and subjecting the precipitate to electrophoresis analysis to determine the level of RNA in the prostate tissue.

27. The method of claim 26, wherein the electrophoresis analysis is conducted on a dilution of the product of an amplification reaction of an RNA extract of the A874 cell line as positive control.

28. A method of reducing or preventing prostate tumor metastasis in a subject in need thereof comprising administering to the subject p75^{NTR} gene or a fragment thereof in an amount effective to prevent or reduce tumor metastasis.

29. A method of reducing or preventing prostate tumor metastasis in a subject in need thereof comprising administering to the subject a p75^{NTR} mRNA stabilizing agent.

30. A method for early diagnosis of prostate cancer comprising determining p75^{NTR} expression levels in prostate tissue of a subject.

31. A method of treatment or prophylaxis of cancer in a subject in need thereof comprising administering to the subject an agent capable of promoting expression of endogenous p75^{NTR} gene in an amount effective to increase tumor suppression and/or tumor apoptosis.

32. A method of treatment or prophylaxis of cancer in a subject in need thereof comprising administering to the subject p75^{NTR} protein in an amount effective to increase tumor suppression and/or tumor apoptosis.

33. The method of claim 32, wherein the p75^{NTR} protein is administered in an amount sufficient to maintain a level of p75^{NTR} which at least partially compensates for the loss of p75^{NTR} mRNA associated with p75^{NTR} mRNA degradation in cancerous or precancerous cells.

34. The method of claim 32, wherein the p75^{NTR} protein is administered directly or in a liposomal delivery system.
